SEQUENCE LISTING (36 sequences)

SEQ ID NO: 1 - protein, 1208 aa, Artificial Sequence
  synthetic sequence
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ

SEQ ID NO: 2 - protein, 651 aa, Artificial Sequence
  synthetic sequence
STIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADGSGLEVLFQGPGSAWSHPQFEKGGGSGGGSGGSAWSHPQFEKGSHHHHHHHHHH

SEQ ID NO: 3 - protein, 634 aa, Artificial Sequence
  synthetic sequence
AENLWVTVYYGVPVWKDAETTLFCASDAKAYETEKHNVWATHCCVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSAITQACPKVSFEPIPIHYCAPAGFAILKCKDKKFNGTGPCPSVSTVQCTHGIKPVVSTQLLLNGSLAEEEVMIRSENITNNAKNILVQFNTPVQINCTRPNNNTRKSIRIGPGQWFYATGDIIGDIRQAHCNVSKATWNETLGKVVKQLRKHFGNNTIIRFANSSGGDLEVTTHSFNCGGEFFYCNTSGLFNSTWISNTSVQGSNSTGSNDSITLPCRIKQIINMWQRIGQAMYAPPIQGVIRCVSNITGLILTRDGGSTNSTTETFRPGGGDMRDNWRSELYKYKVVKIEPLGVAPTRCKRRVVGRRRRRRAVGIGAVFLGFLGAAGSTMGAASMTLTVQARNLLSGIVQQQSNLLRAPECQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 4 - protein, 458 aa, Artificial Sequence
  synthetic sequence
MNRGVPFRHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 5 - protein, 500 aa, Artificial Sequence
  synthetic sequence
  misc_feature (324)..(332): Xaa can be any naturally occurring amino acid
ETGRSIPLGVIHNSALQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTVVSTHHQDTGEESASSGKLGLITNTIAGVAGLITGGRRTRRXXXXXXXXXEAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDGSGYIPEAPRDGQAYVRKDGEWVLLSTFLGTHHHHHH

SEQ ID NO: 6 - protein, 1278 aa, Artificial Sequence
  synthetic sequence
MTARGLALGLLLLLLCPAQVFSQSCVWYGECGIAYGDKRYNCEYSGPPKPLPKDGYDLVQELCPGFFFGNVSLCCDVRQLQTLKDNLQLPLQFLSRCPSCFYNLLNLFCELTCSPRQSQFLNVTATEDYVDPVTNQTKTNVKELQYYVGQSFANAMYNACRDVEAPSSNDKALGLLCGKDADACNATNWIEYMFNKDNGQAPFTITPVFSDFPVHGMEPMNNATKGCDESVDEVTAPCSCQDCSIVCGPKPQPPPPPAPWTILGLDAMYVIMWITYMAFLLVFFGAFFAVWCYRKRYFVSEYTPIDSNIAFSVNASDKGEASCCDPVSAAFEGCLRRLFTRWGSFCVRNPGCVIFFSLVFITACSSGLVFVRVTTNPVDLWSAPSSQARLEKEYFDQHFGPFFRTEQLIIRAPLTDKHIYQPYPSGADVPFGPPLDIQILHQVLDLQIAIENITASYDNETVTLQDICLAPLSPYNTNCTILSVLNYFQNSHSVLDHKKGDDFFVYADYHTHFLYCVRAPASLNDTSLLHDPCLGTFGGPVFPWLVLGGYDDQNYNNATALVITFPVNNYYNDTEKLQRAQAWEKEFINFVKNYKNPNLTISFTAERSIEDELNRESDSDVFTVVISYAIMFLYISLALGHMKSCRRLLVDSKVSLGIAGILIVLSSVACSLGVFSYIGLPLTLIVIEVIPFLVLAVGVDNIFILVQAYQRDERLQGETLDQQLGRVLGEVAPSMFLSSFSETVAFFLGALSVMPAVHTFSLFAGLAVFIDFLLQITCFVSLLGLDIKRQEKNRLDIFCCVRGAEDGTSVQASESCLFRFFKNSYSPLLLKDWMRPIVIAIFVGVLSFSIAVLNKVDIGLDQSLSMPDDSYMVDYFKSISQYLHAGPPVYFVLEEGHDYTSSKGQNMVCGGMGCNNDSLVQQIFNAAQLDNYTRIGFAPSSWIDDYFDWVKPQSSCCRVDNITDQFCNASVVDPACVRCRPLTPEGKQRPQGGDFMRFLPMFLSDNPNPKCGKGGHAAYSSAVNILLGHGTRVGATYFMTYHTVLQTSADFIDALKKARLIASNVTETMGINGSAYRVFPYSVFYVFYEQYLTIIDDTIFNLGVSLGAIFLVTMVLLGCELWSAVIMCATIAMVLVNMFGVMWLWGISLNAVSLVNLVMSCGISVEFCSHITRAFTVSMKGSRVERAEEALAHMGSSVFSGITLTKFGGIVVLAFAKSQIFQIFYFRMYLAMVLLGATHGLIFLPVLLSYIGPSVNKAKSCATEERYKGTERERLLNF

SEQ ID NO: 7 - protein, 497 aa, Artificial Sequence
  synthetic sequence
QNITEEFYQSTCSAVSKGYLSALRTGWYTSVITIELSNIKKIKCNGTDAKIKLIKQELDKYKNAVTELQLLMQSTPATNNQARGSGSGRSLGFLLGVGSAIASGVAVSKVLHLEGEVNKIKSALLSTNKAVVSLSNGVSVLTSKVLDLKNYIDKQLLPIVNKQSCSIPNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDQFDASISQVNEKINQSLAFIRKSDELLSAIGGYIPEAPRDGQAYVRKDGEWVLLSTFL

SEQ ID NO: 8 - protein, 355 aa, Artificial Sequence
  synthetic sequence
MDQFPESVTENFEYDDLAEACYIGDIVVFGTVFLSIFYSVIFAIGLVGNLLVVFALTNSKKPKSVTDIYLLNLALSDLLFVATLPFWTHYLINEKGLHNAMCKFTTAFFFIGFFGSIFFITVISIDRYLAIVLAANSMNNRTVQHGVTISLGVWAAAILVAAPQFMFTKQKENECLGDYPEVLQEIWPVLRNVETNFLGFLLPLLIMSYCYFRIIQTLFSCKNHKKAKAIKLILLVVIVFFLFWTPYNVMIFLETLKLYDFFPSCDMRKDLRLALSVTETVAFSHCCLNPLIYAFAGEKFRRYLYHLYGKCLAVLCGRSVHVDFSSSESQRSRHGSVLSSNFTYHTSDGDALLLL

SEQ ID NO: 9 - protein, 529 aa, Artificial Sequence
  synthetic sequence
MVVILDKRCYCNLLILILMISECSVGILHYEKLSKIGLVKGVTRKYKIKSNPLTKDIVIKMIPNVSDMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDDSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLKLMKQIEDKIEEILSKIYHIENEIARIKKLIGEAPGGIEGR

SEQ ID NO: 10 - protein, 333 aa, Artificial Sequence
  synthetic sequence
MAVRRDSVWKYCWGVLMVLCRTAISKSIVLEPIYWNSSNSKFLPGQGLVLYPQIGDKLDIICPKVDSKTVGQYEYYKVYMVDKDQADRCTIKKENTPLLNCAKPDQDIKFTIKFQEFSPNLWGLEFQKNKDYYIISTSNGSLEGLDNQEGGVCQTRAMKILMKVGQDASSAGSTRNKDPTRRPELEAGTNGRSSTTSPFVKPNPGSSTDGNSAGHSGNNILGSEVALFAGIASGCIIFIVIIITLVVLLLKYRRRHRKHSPQHTTTLSLSTLATPKRSGNNNGSEPSDIIIPLRTADSVFCPHYEKVSGDYGHPVYIVQEMPPQSPANIYYKV

SEQ ID NO: 11 - protein, 395 aa, Artificial Sequence
  synthetic sequence
AHCIGITDRDFIEGVHGGTWVSATLEQDKCVTVMAPDKPSLDISLETVAIDRPAEVRKVCYNAVLTHVKINDKCPSTGEAHLAEENEGDNACKRTYSDRGWGNGCGLFGKGSIVACAKFTCAKSMSLFEVDQTKIQYVIRAQLHVGAKQENWNTDIKTLKFDALSGSQEVEFIGYGKATLECQVQTAVDFGNSYIAEMETESWIVDRQWAQDLTLPWQSGSGGVWREMHHLVEFEPPHAATIRVLALGNQEGSLKTALTGAMRVTKDTNDNNLYKLHGGHVSCRVKLSALTLKGTSYKICTDKMFFVKNPTDTGHGTVVMQVKVSKGAPCRIPVIVADDLTAAINKGILVTVNPIASTNDDEVLIEVNPPFGDSYIIVGRGDSRLTYQWHKEGSS

SEQ ID NO: 12 - protein, 416 aa, Artificial Sequence
  synthetic sequence
MIRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGKHHHHHH

SEQ ID NO: 13 - protein, 404 aa, Artificial Sequence
  synthetic sequence
MSDSKEPRLQQLGLLEEEQLRGLGFRQTRGYKSLAGCLGHGPLVLQLLSFTLLAGLLVQVSKVPSSISQEQSRQDAIYQNLTQLKAAVGELSEKSKLQEIYQELTQLKAAVGELPEKSKLQEIYQELTRLKAAVGELPEKSKLQEIYQELTWLKAAVGELPEKSKMQEIYQELTRLKAAVGELPEKSKQQEIYQELTRLKAAVGELPEKSKQQEIYQELTRLKAAVGELPEKSKQQEIYQELTQLKAAVERLCHPCPWEWTFFQGNCYFMSNSQRNWHDSITACKEVGAQLVVIKSAEEQNFLQLQSSRSNRFTWMGLSDLNQEGTWQWVDGSPLLPSFKQYWNRGEPNNVGEEDCAEFSGNGWNDDKCNLAKFWICKKSAASCSRDEEQFLSPAPATPNPPPA

SEQ ID NO: 14 - protein, 401 aa, Artificial Sequence
  synthetic sequence
PVMCLLANTTFPCSQPPCTPCCYEKEPEETLRMLEDNVMRPGYYQLLQASLTCSPHRQRESTKDNFNVYKATRPYLAHCPDCGEGHSCHSPVALERIRNEATDGTLKIQVSLQIGIKTDDSHDWTKLRYMDNHMPADAERAGLFVRTSAPCTITGTMGHFILARCPKGETLTVGFTDSRKISHSCTHPFHHDPPVIGREKFHSRPQHGKELPCSTYVQSTAATTEEIEVHMPPDTPDRTLMSQQSGNVKITVNGQTVRYKCNCGGSNEGLTTTDKVINNCKVDQCHAAVTNHKKWQYNSPLVPRNAELGDRKGKIHIPFPLANVTCRVPKARNPTVTYGKNQVIMLLYPDHPTLLSYRNMGEEPNYQEEWVMHKKEVVLTVPTEGLEVTWGNNEPYKYWPQ

SEQ ID NO: 15 - protein, 393 aa, Artificial Sequence
  synthetic sequence
GGYEHVTVIPNTVGVPYKTLVNRPGYSPMVLEMELLSVTLEPTLSLDYITCEYKTVIPSPYVKCCGTAECKDKNLPDYSCKVFTGVYPFMWGGAYCFCDAENTQLSEAHVEKSESCKTEFASAYRAHTASASAKLRVLYQGNNITVTAYANGDHAVTVKDAKFIVGPMSSAWTPFDNKIVVYKGDVYNMDYPPFGAGRPGQFGDIQSRTPESKDVYANTQLVLQRPAAGTVHVPYSQAPSGFKYWLKERGASLQHTAPFGCQIATNPVRAVNCAVGNMPISIDIPEAAFTRVVDAPSLTDMSCEVPACTHSSDFGGVAIIKYAASKKGKCAVHSMTNAVTIREAEIEVEGNSQLQISFSTALASAEFRVQVCSTQVHCAAECHPPKDHIVNYP

SEQ ID NO: 16 - protein, 442 aa, Artificial Sequence
  synthetic sequence
MALPSRILLWKLVLLQSSAVLLHSGSSVPAAAGSSVVSESAVSWEAGARAVLRCQSPRMVWTQDRLHDRQRVLHWDLRGPGGGPARRLLDLYSAGEQRVYEARDRGRLELSASAFDDGNFSLLIRAVEETDAGLYTCNLHHHYCHLYESLAVRLEVTDGPPATPAYWDGEKEVLAVARGAPALLTCVNRGHVWTDRHVEEAQQVVHWDRQPPGVPHDRADRLLDLYASGERRAYGPLFLRDRVAVGADAFERGDFSLRIEPLEVADEGTYSCHLHHHYCGLHERRVFHLTVAEPHAEPPPRGSPGNGSSHSGAPGPDPTLARGHNVINVIVPESRAHFFQQLGYVLATLLLFILLLVTVLLAARRRRGGYEYSDQKSGKSKGKDVNLAEFAVAAGDQMLYRSEDIQLDYKNNILKERAELAHSPLPAKYIDLDKGFRKENCK

SEQ ID NO: 17 - protein, 497 aa, Artificial Sequence; identical to SEQ ID NO: 7
  synthetic sequence

SEQ ID NO: 18 - protein, 355 aa, Artificial Sequence; identical to SEQ ID NO: 8
  synthetic sequence

SEQ ID NO: 19 - protein, 529 aa, Artificial Sequence; identical to SEQ ID NO: 9
  synthetic sequence

SEQ ID NO: 20 - protein, 333 aa, Artificial Sequence; identical to SEQ ID NO: 10
  synthetic sequence

SEQ ID NO: 21 - protein, 542 aa, Artificial Sequence
  synthetic sequence
MSWKVVIIFSLLITPQHGLKESYLEESCSTITEGYLSVLRTGWYTNVFTLEVGDVENLTCADGPSLIKTELDLTKSALRELRTVSADQLAREEQIENPRRRRFVLGAIALGVATAAAVTAGVAIAKTIRLESEVTAIKNALKKTNEAVSTLGNGVRVLATAVRELKDFVSKNLTRAINKNKCDIPDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSSAEKGNTSGRENLYFQGGGGSGYIPEAPRDGQAYVRKDGEWVLLSTFLGGIEGRHHHHHH

SEQ ID NO: 22 - protein, 438 aa, Artificial Sequence
  synthetic sequence
MGQIVTMFEALPHIIDEVINIVIIVLIIITSIKAVYNFATCGILALVSFLFLAGRSCGMYGLNGPDIYKGVYQFKSVEFDMSHLNLTMPNACSANNSHHYISMGSSGLELTFTNDSILNHNFCNLTSAFNKKTFDHTLMSIVSSLHLSIRGNSNHKAVSCDFNNGITIQYNLSFSDPQSAISQCRTFRGRVLDMFRTAFGGKYMRSGWGWAGSDGKTTWCSQTSYQYLIIQNRTWENHCRYAGPFGMSRILFAQEKTKFLTRRLAGTFTWTLSDSSGVENPGGYCLTKWMILAAELKCFGNTAVAKCNVNHDEEFCDMLRLIDYNKAALSKFKQDVESALHVFKTTVNSLISDQLLMRNHLRDLMGVPYCNYSKFWYLEHAKTGETSVPKCWLVTNGSYLNETHFSDQIEQEADNMITEMLRKDYIKRQGSTPLALMD

SEQ ID NO: 23 - protein, 895 aa, Artificial Sequence
  synthetic sequence
MRMSVGLSLLLPLSGRTFLLLLSVVMAQSHWPSEPSEAVRDWENQLEASMHSVLSDLHEAVPTVVGIPDGTAVVGRSFRVTIPTDLIASSGDIIKVSAAGKEALPSWLHWDSQSHTLEGLPLDTDKGVHYISVSATRLGANGSHIPQTSSVFSIEVYPEDHSELQSVRTASPDPGEVVSSACAADEPVTVLTVILDADLTKMTPKQRIDLLHRMRSFSEVELHNMKLVPVVNNRLFDMSAFMAGPGNAKKVVENGALLSWKLGCSLNQNSVPDIHGVEAPAREGAMSAQLGYPVVGWHIANKKPPLPKRVRRQIHATPTPVTAIGPPTTAIQEPPSRIVPTPTSPAIAPPTETMAPPVRDPVPGKPTVTIRTRGAIIQTPTLGPIQPTRVSEAGTTVPGQIRPTMTIPGYVEPTAVATPPTTTTKKPRVSTPKPATPSTDSTTTTTRRPTKKPRTPRPVPRVTTKVSITRLETASPPTRIRTTTSGVPRGGEPNQRPELKNHIDRVDAWVGTYFEVKIPSDTFYDHEDTTTDKLKLTLKLREQQLVGEKSWVQFNSNSQLMYGLPDSSHVGKHEYFMHATDKGGLSAVDAFEIHVHRRPQGDRAPARFKAKFVGDPALVLNDIHKKIALVKKLAFAFGDRNCSTITLQNITRGSIVVEWTNNTLPLEPCPKEQIAGLSRRIAEDDGKPRPAFSNALEPDFKATSITVTGSGSCRHLQFIPVVPPRRVPSEAPPTEVPDRDPEKSSEDDVYLHTVIPAVVVAAILLIAGIIAMICYRKKRKGKLTLEDQATFIKKGVPIIFADELDDSKPPPSSSMPLILQEEKAPLPPPEYPNQSVPETTPLNQDTMGEYTPLRDEDPNAPPYQPPPPFTAPMEGKGSRPKNMTPYRSPPPYVPP

SEQ ID NO: 24 - protein, 895 aa, Artificial Sequence; identical to SEQ ID NO: 23
  synthetic sequence

SEQ ID NO: 25 - protein, 904 aa, Artificial Sequence
  synthetic sequence
MHQGAPSWGRRWFVVWALLGLTLGVLVASAAPSSPGTPGVAAATQAANGGPATPAPPALGAAPTGDPKPKKNKKPKNPTPPRPAGDNATVAAGHATLREHLRDIKAENTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYLANGGFLIAYQPLLSNTLAELYVREHLREQSRKPPNPTPPPPGASANASVERIKTTSSIEFARLQFTYNHIQRPVNDMLGRVAIAWCELQNHELTLWNEARKLNPNAIASVTVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAAMFAGLGAFFEGMGDLGRAVGKVVMGIVGGVVSAVSGVSSFMSNPFGALAVGLLVLAGLAAAFFAFRYVMRLQSNPMKALYPLTTKELKNPTNPDASGEGEEGGDFDEAKLAEAREMIRYMALVSAMERTEHKAKKKGTSALLSAKVTDMVMRKRRNTNYTQVPNKDGDADEDDL

SEQ ID NO: 26 - protein, 389 aa, Artificial Sequence
  synthetic sequence
MGQNLSTSNPLGFFPDHQLDPAFRANTANPDWDFNPNKDTWPDANKVGAGAFGLGFTPPHGGLLGWSPQAQGILQTLPANPPPASTNRQSGRQPTPLSPPLRNTHPQAMQWNSTTFHQTLQDPRVRGLYFPAGGSSSGTVNPVLTTASPLSSIFSRIGDPALNMENITSGFLGPLLVLQAGFFLLTRILTIPQSLDSWWTSLNFLGGTTVCLGQNSQSPTSNHSPTSCPPTCPGYRWMCLRRFIIFLFILLLCLIFLLVLLDYQGMLPVCPLIPGSSTTSTGPCRTCMTTAQGTSMYPSCCCTKPSDGNCTCIPIPSSWAFGKFLWEWASARFSWLSLLVPFVQWFVGLSPTVWLSVIWMMWYWGPSLYSILSPFLPLLPIFFCLWVYI

SEQ ID NO: 27 - protein, 349 aa, Artificial Sequence
  synthetic sequence
MEAHNASAPFNFTLPPNFGKRPTDLALSVILVFMLFFIMLSLGCTMEFSKIKAHLWKPKGLAIALVAQYGIMPLTAFVLGKVFRLKNIEALAILVCGCSPGGNLSNVFSLAMKGDMNLSIVMTTCSTFCALGMMPLLLYIYSRGIYDGDLKDKVPYKGIVISLVLVLIPCTIGIVLKSKRPQYMRYVIKGGMIIILLCSVAVTVLSAINVGKSIMFAMTPLLIATSSLMPFIGFLLGYVLSALFCLNGRCRRTVSMETGCQNVQLCSTILNVAFPPEVIGPLFFFPLLYMIFQLGEGLLLIAIFWCYEKFKTPKDKTKMIYTAATTEETIPGALGNGTYKGEDCSPCTA

SEQ ID NO: 28 - protein, 235 aa, Artificial Sequence
  synthetic sequence
NQLINTNGSWHLNRTALNCNDSLNTGWLAGLFYHHKFDSSGCPERMASCRPLTDFDQGWGPISHANGSGPDGSSGCWHYPPRPCGIVPAKTVCGPVYCFTPSPVVVGTTDRAGAPAYNWGENDTDVFVLNNTRPPLGNWFGCTWMNSTGFTKACGAPPCAIGGVGDKTLYCPTDCFRKHPEATYSRCGSGPWITPRCLVDYPYRLWHYPCTINYTVFKIRMYVGGVEHRLEAACN

SEQ ID NO: 29 - protein, 236 aa, Artificial Sequence
  synthetic sequence
MGVEGCTKCIKYLLFVFNFVFWLAGGVILGVALWLRHDPQTTNLLYLELGDKPAPNTFYVGIYILIAVGAVMMFVGFLGCYGAIQESQCLLGTFFTCLVILFACEVAAGIWGFVNKDQIAKDVKQFYDQALQQAVVDDDANNAKAVVKTFHETLDCCGSSTLTALTTSVLKNNLCPSGSNIISNLFKEDCHQKIDDLFSGKLYLIGIAAIVVAVIMIFEMILSMVLCCGIRNSSVY

SEQ ID NO: 30 - protein, 1287 aa, Artificial Sequence
  synthetic sequence
MFVFLVLLPLVSSQCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHGSASSVASQSIIAYTMSLGAENSVAYSNNSIAIPINFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILARLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTHNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGTSLEVLFQGPGHHHHHHHHHHSAWSHPQFEKGGGSGGGGSGGSAWSHPQFEK

SEQ ID NO: 31 - protein, 1285 aa, Artificial Sequence
  synthetic sequence
MFVFLVLLPLVSSQCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHISYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGHHHHHHHHSAWSHPQFEKGGGSGGGGSGGSAWSHPQFEK

SEQ ID NO: 32 - protein, 1286 aa, Artificial Sequence
  synthetic sequence
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGHHHHHHHHSAWSHPQFEKGGGSGGGGSGGSAWSHPQFEK

SEQ ID NO: 33 - protein, 1290 aa, Artificial Sequence
  synthetic sequence
MFVFLVLLPLVSSQCVNFTNRTQLPSAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNYPFLGVYYHKNNKSWMESEFRVYSSANNCTFEYVSQPFLMDLEGKQGNFKNLSEFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAAIKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASFVNIQKEIDRLNEVAKNLNESLIDLQELGKYEQGSGYIPEAPRDGQAYVRKDGEWVLLSTFLGRSLEVLFQGPGHHHHHHHHHHSAWSHPQFEKGGGSGGGGSGGSAWSHPQFEK

SEQ ID NO: 34 - protein, 1288 aa, Artificial Sequence
  synthetic sequence
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMKSEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVQGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAHEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGHHHHHHHHSAWSHPQFEKGGGSGGGGSGGSAWSHPQFEK

SEQ ID NO: 35 - protein, 1281 aa, Artificial Sequence
  synthetic sequence
MFVFLVLLPLVSSQCVNLTTRTQLPPAYTNSFTRGVYYPDKVFRSSVLHSTQDLFLPFFSNVTWFHAIHVSGTNVIKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHNSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYQYRLFRKSNLKPFERDISTEIYQAGSTPCNGVEGFNCYSPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLNVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGHHHHHHHHSAWSHPQFEKGGGSGGGGSGGSAWSHPQFEK

SEQ ID NO: 36 - protein, 1289 aa, Artificial Sequence
  synthetic sequence
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASIEKSNIIRGWIFGTTLDSKTQSLLIVNNATNVVIKVCEFQFCNDPFLGVTSNHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATKFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGHHHHHHHHSAWSHPQFEKGGGSGGGGSGGSAWSHPQFEK

(19) United States
(12) Patent Application Publication
(10) Pub. No.: US 2023/0393135 A1
(43) Pub. Date: Dec. 7, 2023

(54) Cell-Free Method For The Quantitative Measurement Of Virus Neutralizing Antibodies

(71) Applicant: ECOLE POLYTECHNIQUE FEDERALE DE LAUSANNE (EPFL), Lausanne (CH)

(72) Inventors: Didier TRONO, Vufflens-Chateau (CH); Priscilla TURELLI, Echandens-Denges (CH); Giuseppe PANTALEO, Pully (CH); Craig FENWICK, Lausanne (CH)

(21) Appl. No.: 18/032,920

(22) PCT Filed: Nov. 2, 2021
(86) PCT No.: PCT/EP2021/080399

§ 371 Date: Apr. 20, 2023

(30) Foreign Application Priority Data
Nov. 2, 2020 (EP) 20205298.1
Apr. 7, 2021 (EP) 21167269.6

(51) Int. Cl. G01N 33/569

(52) U.S. Cl. CPC G01N 33/56983; G01N 2469/20; G01N 2333/165; G01N 2500/02

(57) ABSTRACT

The invention provides an in vitro method for the cell-free quantification of virus neutralizing activity, such as the SARS-CoV-2 neutralizing activity, of biological samples and kit for carrying out the method. The invention also provides a method of screening biological samples or collections of molecules to identify neutralizing antibodies or antiviral drugs.

### FIELD OF THE INVENTION

The invention provides an in vitro method for the cell-free quantification of virus neutralizing activity, such as the SARS-CoV-2 neutralizing activity, of biological samples and kit for carrying out the method. The invention also provides a method of screening biological samples or collections of molecules to identify neutralizing antibodies or antiviral drugs.

### BACKGROUND OF THE INVENTION

Many viruses are enveloped with a lipid bilayer and infect their target cells by inducing the fusion of the viral and cell membranes. The viral envelope protein is the key factor that induces this fusion reaction to allow viral entry. This virus-encoded envelope protein mediates attachment to the cell surface and induces the subsequent membrane fusion reaction. Although there are efficient vaccines against some pathogenic human or animal viruses, for the majority there is no prophylactic or therapeutic treatment. The known vaccines typically work by eliciting antibodies that block entry of the pathogen into cells, which in the case of enveloped viruses involves antibody binding to the viral envelope proteins.

The presence of specific antibodies in body fluids such as serum or mucosal secretions, whether triggered by a natural infection or a vaccine, is a marker of adaptive immune responses against a virus. Neutralizing antibodies stand out amongst immunoglobulins generated in these contexts owing to their ability to block the infectivity of the virus. Neutralizing antibodies are protective antibodies that are naturally produced by our humoral immune system. To elicit their antiviral activity, neutralizing antibodies bind to the surface epitopes of viral particles to prevent their entry into a host cell. As a consequence, they are generally interpreted as indicating some degree or protection against (re)infection.

The measurement of neutralizing antibodies, for example against SARS-CoV-2, is of paramount importance within the context of epidemics and pandemics, such as the COVID-19 pandemics, as it allows one to identify individuals likely resistant to infection either because they were previously infected and mounted a protective immune response or because they received an effective vaccine. This parameter is thus of primary relevance for monitoring the degree and duration of immune protection conferred by viral infection, such as SARS-CoV-2 infection, and to assess the value of vaccines, such as SARS-CoV-2 vaccines. Accordingly, quantification of neutralizing antibodies, such as SARS-CoV-2 neutralizing antibodies, stand to be performed repetitively in billions of individuals in the months and years to come.

SARS-CoV-2 neutralizing antibodies are currently detected by assessing the ability of a biological sample (e.g. serum sample) to prevent in tissue culture the infection of cells (e.g. Vero cells) expressing the ACE2 SARS-CoV-2 receptor by either the virus itself (be it wild-type or some modified variant, e.g. expressing GFP) or by particles from some other virus or viral derivative (e.g. lentiviral vectors) pseudotyped with the SARS-CoV-2 spike protein, responsible for mediating viral entry. SARS-CoV-2 neutralizing antibodies indeed block viral infectivity by recognizing the viral spike protein usually over its receptor binding domain (RBD), thereby precluding its ability to bind the ACE2 receptor. These cell-based neutralization assays suffer from several shortcomings: they are technically demanding, requiring either a biosafety level 3 (BLS3) facilities for SARS-CoV-2 and derivatives or a biosafety level 2 (BSL2) facilities for viral pseudotypes; they are time-consuming, typically requiring between 24 and 48 hrs for read-out, which is based on the efficiency of limited dilutions of a biological sample (e.g. serum) to block SARS-CoV-2 or spike-pseudotyped vector particles-induced cytopathic effects or reporter gene expression, respectively; whereas viral pseudotypes are attractive owing to their milder degree of biohazard, their production is technically demanding (typically requiring the transfection of producer cells with several plasmids), and their relative infectivity is far lower than that of wild type virus because their components get assembled at the plasma membrane whereas SARS-CoV-2 normally loads its spike protein in special subdomains of the endoplasmic reticulum-Golgi intermediate compartment (ERGIC). Due their low (and often erratic) infectivity, spike-pseudotyped vector particles are far easier to neutralize than wild-type SARS-CoV-2, leading to unreliable estimations of the neutralizing titers of samples.

Measuring the neutralizing activity of virus-specific antibodies is further complicated by the continuous emergence of SARS-CoV-2 variants. Some of these variants are of particular clinical relevance, such as ones with mutations in and around the receptor binding domain (RBD) of the S protein, resulting in increased affinity for the ACE2 receptor or reduced recognition by neutralizing antibodies. For instance, the B.1.351 (also called 501Y.V2 or South African variant of concern (VOC)) has been found largely to escape immunity induced by some COVID-19 vaccines, and the closely related P.1 (also called 501Y.V3, B.1.1.28 or Brazilian VOC) to be responsible for large numbers of reinfections in the Manaus region.

An in vitro cell-free assay relying on the binding of monomeric fragment of spike (i.e. a receptor binding domain (RBD)) to ACE2 was recently described and made commercially available (Tan, C. W., Chia, W. N., Qin, X. et al., “A SARS-CoV-2 surrogate virus neutralization test based on antibody-mediated blockage of ACE2-spike protein-protein interaction”, Nat Biotechnol 38, 1073-1078, 2020). However, its performance is not sufficient for quantifying neutralizing antibodies.

Therefore, there is still a need for neutralizing antibodies quantification method that would allow an accurate quantification of neutralizing antibodies in biological samples collected from patients subjected to antibody or vaccine treatments or to viral infections.

### SUMMARY OF THE INVENTION

An aspect of the present invention provides a method for quantifying virus neutralizing antibodies in a biological sample of a subject, wherein the virus is enveloped virus and wherein the method comprises
a) providing at least one prefusion viral protein(s), that is/are stabilized in a prefusion conformation and that is/are reactive with the neutralizing antibodies, and coupling said prefusion viral protein(s) to at least one solid support,
b) obtaining a biological sample from a subject,
c) contacting the at least one prefusion viral protein(s) coupled to the at least one solid support with the biological sample to provide a mixture,
d) contacting the mixture with one or more cell receptor proteins that bind to the at least one prefusion viral protein(s) or one or more competing antibodies that bind to the at least one prefusion viral protein(s),
e) detecting and quantifying neutralizing antibodies in the biological sample.

Another aspect of the present invention provides a kit comprising
at least one prefusion viral protein(s), that is stabilized in a prefusion conformation and that is reactive with neutralizing antibodies,
one or more cell receptor proteins that bind to the at least one prefusion viral protein or one or more competing antibody that binds to the at least one prefusion viral protein(s), and
instructions for use.

A further aspect of the present invention provides a method for measuring the ability of an agent to block entry of a virus into a cell susceptible to virus infection and thereby to inhibit infection of the cell with the virus, wherein the virus is enveloped virus and wherein the method comprises
a) providing at least one prefusion viral protein, that is stabilized in a prefusion conformation and/or that is reactive with the virus neutralizing antibodies, and coupling said prefusion viral protein to at least one solid support,
b) contacting the at least one prefusion viral protein coupled to the at least one solid support with the agent to provide a mixture,
c) contacting the mixture with at least one cell receptor protein that binds to the at least one prefusion viral protein,
d) measuring the binding of the cell receptor to the prefusion viral protein to provide an indirect measure of the ability of the agent to inhibit entry of the virus into the cell.

### BRIEF DESCRIPTION OF THE FIGURES

FIG. 1 shows competitive binding assay to identify sera with antibodies that block the interaction between the SARS-CoV-2 spike protein and its ACE-2 receptor. Beads adorned with spike trimers in prefusion configuration are incubated with serum samples of patients at increasing dilutions. Recombinant ACE-2 protein is added and incubated for a further 30 minutes. The beads are washed and bound ACE-2 is detected with a PE-labelled secondary antibody. Healthy donor sera have no effect on the Spike/ACE-2 interaction, whereas sera containing neutralising anti-Spike antibodies have the capacity to block the Spike/ACE-2 interaction.

FIG. 2 shows the results of a neutralization assay performed by measuring the ability of patients sera to block infection of VeroE6 cells by wild-type SARS-CoV-2 viral particles.

FIG. 3 shows comparison of neutralization assay according to the method of the invention and live SARS-CoV-2 cytopathic effect assay. Spike/ACE-2 competitive binding assay accurately reflects neutralization potency.

FIG. 4 shows comparison of methods based on Spike and RBD fragment.

FIG. 5 shows comparison of method of the invention (surrogate neutralization assay) and commercial assay from GeneScript.

FIG. 6 shows outline and validation of a cell-free SARS-CoV-2 S protein trimer/ACE2 surrogate neutralization assay: (a) Schematic outline of the S3/ACE2 neutralization assay. Anti-SARS-CoV-2 serum antibodies are monitored for their capacity in blocking the S3/ACE2 interaction. ACE2 binding to S protein was detected though use of a fluorescently labelled secondary antibody and the signal intensities are inversely proportional to the neutralizing potential of the anti-S protein antibodies. (b) Serum dilution IC₅₀ values were calculated for 104 healthy adult donor samples collected prior to November 2019 (pre-COVID-19 pandemic). The mean IC₅₀ values and SD were used to establish a lower limit cutoff of 50 indicated by the dashed line (mean IC₅₀ of 12.5+4×9.0 SD=50 serum dilution). (c) Representative concentration response curves for ten healthy donor serum samples and (d) ten SARS-CoV-2 seropositive donors with varying levels of anti-S protein IgG antibody. Mean±s.d. shown in graph b. S protein/ACE2 structure was generated with PDB 7a98.

FIG. 7 shows anti-S protein IgG and IgA levels in the seropositive donor groups. Anti-S protein IgG (a) and IgA (b) antibody levels measured in the serum samples of different donor groups using a Luminex Spike trimer serological assay. Donors were seropositive for anti-S protein IgG and/or IgA antibodies with positivity cutoffs of 4 and 6.5 ratios over a standard negative control, respectively (red dashed line in (a) and (b). Donor groups consist of seropositive volunteers, randomly selected individuals from the general population and donors that were in contact with a RT-PCR confirmed infected subject (n=111) with reduced level of symptoms and symptomatic RT-PCR positive donors that were either hospitalized COVID-19 patients or not (n=95). Boxplots shown mean and the 95% interquartile range.

FIG. 8 shows cross-validation of a cell-free SARS-CoV-2 S protein trimer/ACE2 surrogate neutralization assay: (a) Cross-validation studies between S3/ACE2 surrogate neutralization assay and the “gold standard” SARS-CoV-2 cytopathic effect (CPE) neutralization assay. Seropositive donors (n=206) with varying levels of anti-S protein antibodies were selected for comparison of the assays. Donors consisted of COVID-19 hospitalized patients (n=31), symptomatic infected donors with RT-PCR-documentation (n=64) and other seropositive donors identified through random sampling, volunteers or contact with a confirmed SARS-CoV-2 infected individual in a Swiss population serological survey (n=111). (b) Correlation between the live SARS-CoV-2 virus cytopathic effect and S protein pseudotyped neutralization assays. A group of 74 samples from S protein seropositive donors were compared in the live virus CPE and S protein pseudotyped virus cell based neutralization assays. Correlation between the two neutralization assays is represented by the black dashed line and the 20 serum dilution IC₅₀ cutoff for positivity in the CPE assay is shown with the blue dashed line.

FIG. 9 shows multiplexed analysis of antibody and serum neutralizing activity against VOC S protein mutations. The surrogate neutralization assay was performed with two panels of S3 coupled beads consisting of the 2019-nCov S protein and S mutations produced with one or more amino acid substitutions or deletions. (a) Concentration response curves of the REGN10933 therapeutic antibody evaluated against S protein mutations from three viral variants in the S3/ACE2 assay. (b-c) Serum dilutions from RT-PCR positive donors 3506 and 9504 (b) or 1034 and 5537 (c) in the S3-ACE2 assay with two separate panels of S3 coupled beads. (c) Spider plot and heatmap showing IC50 serum dilutions for donors 1034 and 5537 serums samples against the indicated S protein mutations found in variants of concern including B.1.1.7 and P1. In these graphs, green background corresponds to an IC50 dilution >100 for strong blocking of the S3/ACE2 interaction, yellow background for moderate blocking with IC₅₀ dilutions from 50 to 100 and red background for low to no blocking activity against the indicated S protein mutant.

FIG. 10 shows multiplexed analysis of COVID-19 patient serum samples for neutralizing activity against variant of concern S protein mutations. Serum samples from 59 COVID-19 hospitalized patients were profiled in the multiplexed S3/ACE2 surrogate neutralization assay performed in parallel with the 2019-nCov S protein and three S mutants produced with one or more amino acid substitutions or deletions. S³/ACE2 serum dilution IC₅₀ values for each of the S proteins were calculated for samples from the 28 COVID-19 hospitalized donors that remained on the wards (non-ICU, closed symbols) and for 31 COVID-19 patients that were critically ill, requiring treatment by the intensive care unit (ICU, open symbols). Dashed red line indicates the 50 serum dilution IC₅₀ value cut-off in the S3/ACE2 assay that corresponds to neutralizing antibody levels needed for near complete (>99%) neutralization in the live virus CPE assay. Percentages of serum samples above this cut-off are shown for non-ICU and ICU donors for each of the S proteins tested. Median values are shown as a red bar for each of the data sets. Statistical analysis was performed using the non-parametric Mann-Whitney t-test and showed that ICU patients had higher levels of neutralizing antibodies compared to non-ICU patients (P=0.0127 to 0.0425) when compared for the same S protein. In comparisons between S proteins, reduced levels of neutralizing antibodies against the S protein with K417N/E484K/N501Y mutations were strongly significant (P<0.0001) relative to the 2019-nCoV S protein.

FIG. 11 shows selection of a positive cutoff values in the live virus CPE neutralization assay. Simulated data generated to illustrate that maximum viral inhibitions of 95% and 99% would be obtained with a serum dilution IC₅₀ of 20 using either Hill slope values of 1.0 and 1.5, respectively.

FIG. 12 shows neutralization activity of donor sera tested against pseudotyped viruses produced with different S protein mutations. Concentration response curves generated for donor 3506 (a) and 9504 (b) serum samples in the SARS-CoV-2 S protein pseudoviral assay using either the 2019-nCoV D614G, S477N/D614G or E484K D614G S protein pseudotyped particles. Graphs show the mean of n=2±s.d.

### DETAILED DESCRIPTION OF THE INVENTION

All publications, patent applications, patents, and other references mentioned herein are incorporated by reference in their entirety. The publications and applications discussed herein are provided solely for their disclosure prior to the filing date of the present application. Nothing herein is to be construed as an admission that the present invention is not entitled to antedate such publication by virtue of prior invention. In addition, the materials, methods, and examples are illustrative only and are not intended to be limiting.

In the case of conflict, the present specification, including definitions, will control. Unless defined otherwise, all technical and scientific terms used herein have the same meaning as is commonly understood by one of skill in the art to which the subject matter herein belongs. As used herein, the following definitions are supplied in order to facilitate the understanding of the present invention.

The term “comprise” is generally used in the sense of include, that is to say permitting the presence of one or more features or components. Also as used in the specification and claims, the language “comprising” can include analogous embodiments described in terms of “consisting of” and/or “consisting essentially of”.

As used in the specification and claims, the singular form “a”, “an” and “the” include plural references unless the context clearly dictates otherwise.

As used in the specification and claims, the term “and/or” used in a phrase such as “A and/or B” herein is intended to include “A and B”, “A or B”, “A”, and “B”.

As used herein, the terms “subject” and “patient” are well-recognized in the art, and, are used herein to refer to a mammal, and most preferably a human. In some embodiments, the subject is a subject in need of treatment and/or a subject being infected by a virus and/or a subject that has received a vaccine treatment. The term does not denote a particular age or sex. Thus, individuals of all ages, from newborn to adult, whether male or female, are intended to be covered.

As used herein, a “neutralizing antibody” is one that neutralizes the ability of a virus, such as SARS-CoV-2 virus to initiate and/or perpetuate an infection in a host and/or in target cells in vitro. Typically, the neutralizing antibody recognizes an antigen from a virus, e.g., Spike protein of SARS-CoV-2 virus.

As used herein, the terms “native protein” or “native sequence” refers to a protein, a polypeptide, or a sequence that has not been modified, for example, by selective mutation. For example, selective mutation to focus the antigenicity of the antigen to a target epitope, or to introduce a disulfide bond into a protein that does not occur in the native protein. Native protein or native sequence are also referred to as wild-type protein or wild-type sequence.

As used herein, the term “recombinant”, such as a recombinant amino acid molecule or a recombinant nucleic acid molecule is one that has a sequence that is naturally occurring or a sequence that is not naturally occurring, for example, includes one or more amino acid or nucleic acid substitutions, deletions or insertions, and/or has a sequence that is made by an artificial combination of two otherwise separated segments of sequence. This artificial combination can be accomplished by chemical synthesis or, more commonly, by the artificial manipulation of isolated segments of amino acids or nucleic acids, for example, by genetic engineering techniques. A recombinant protein is one that has a sequence that is not naturally occurring or has a sequence that is made by an artificial combination of two otherwise separated segments of sequence. In several embodiments, a recombinant protein is encoded by a heterologous (for example, recombinant) nucleic acid that has been introduced into a host cell, such as a bacterial or eukaryotic cell, or into the genome of a recombinant virus.

A “multiplex assay” or “multiplex method” is an assay or a method that simultaneously measures the levels of more than one analyte, such as more than one neutralizing antibody, in a single sample. For example, in the current disclosure, a multiplex assay or method is an assay or a method capable of measuring more than one different neutralizing antibodies in one biological sample. An advantage of the multiplex methods and kits herein disclosed is the small size of biological sample that is required. A second advantage is the ability to detect the presence and concentration of numerous neutralizing antibodies simultaneously in one reaction container. A third advantage is the ability to quantify neutralizing antibodies in a biological sample and a fourth advantage is the ability to directly compare neutralizing antibody titers and/or profiles of an individual before and after a viral infection and/or vaccination and to follow neutralizing antibody titers and/or profiles several months or years after the viral infection and/or vaccination.

For nucleotide and amino acid sequences, the term “identical” or “identity” indicates the degree of identity between two nucleic acid or two amino acid sequences when optimally aligned and compared with appropriate insertions, substitutions or deletions. The percent identity between two sequences is a function of the number of identical positions shared by the sequences (i.e., % identity=number of identical positions/total number of positions multiplied by 100), taking into account the number of gaps, and the length of each gap, which need to be introduced for optimal alignment of the two sequences. The comparison of sequences and determination of percent identity between two sequences can be accomplished using a mathematical algorithm, as described below. Percent identity between a query nucleic acid sequence and a subject nucleic acid sequence is the “Identities” value, expressed as a percentage, which is calculated by the BLASTN algorithm when a subject nucleic acid sequence has 100% query coverage with a query nucleic acid sequence after a pair-wise BLASTN alignment is performed. Such pair-wise BLASTN alignments between a query nucleic acid sequence and a subject nucleic acid sequence are performed by using the default settings of the BLASTN algorithm available on the National Center for Biotechnology Institute's website with the filter for low complexity regions turned off. Percent identity between a query amino acid sequence and a subject amino acid sequence is the “Identities” value, expressed as a percentage, which is calculated by the BLASTP algorithm when a subject amino acid sequence has 100% query coverage with a query amino acid sequence after a pair-wise BLASTP alignment is performed. Such pair-wise BLASTP alignments between a query amino acid sequence and a subject amino acid sequence are performed by using the default settings of the BLASTP algorithm available on the National Center for Biotechnology Institute's website with the filter for low complexity regions turned off. Importantly, a query amino acid sequence may be described by an amino acid sequence identified in one or more claims herein.

In the context of the present invention, a neutralizing antibody is an immunoglobulin, antigen-binding fragment, or derivative thereof, that specifically binds and recognizes an analyte (antigen) such as a surface protein (for example a coronavirus S protein), an antigenic fragment thereof, or a dimer or multimer of the antigen. The term “antibody” is used herein in the broadest sense and encompasses various antibody structures, including but not limited to monoclonal antibodies, polyclonal antibodies, multispecific antibodies (e.g., bispecific antibodies), and antibody fragments, so long as they exhibit the desired antigen-binding activity. Non-limiting examples of antibodies include, for example, intact immunoglobulins and variants and fragments thereof that retain binding affinity for the antigen. Examples of antibody fragments include but are not limited to Fv, Fab, Fab′, Fab′-SH, F(ab′)2; diabodies; linear antibodies; single-chain antibody molecules (e.g. scFv); and multispecific antibodies formed from antibody fragments. Antibody fragments include antigen binding fragments either produced by the modification of whole antibodies or those synthesized de novo using recombinant DNA methodologies.

Determining the level of immune protection conferred by prior virus infection, such as SARS-CoV-2 infection, vaccination or the prophylactic administration of monoclonal antibodies is of paramount importance for informing individuals about their susceptibility to the virus, for adapting prophylactic measures to the evolving viral strains circulating in the population and, ultimately, for example, for controlling the COVID-19 pandemic. While T-cell-based responses may contribute to this immunity, neutralizing antibodies likely play a primary role in this process as they do for other acute viral infections and represent the best available surrogate marker of protection. However, serum levels of neutralizing antibodies, such as SARS-CoV-2 neutralizing antibodies, are so far only rarely measured owing to overwhelming technical difficulties and biosafety requirements, which preclude any sort of routine procedure or significant scale-up.

The present disclosure presents a cell-free quantitative neutralization assay based on the competitive inhibition of trimeric SARS-CoV-2 Spike protein (SARS-CoV-2 protein) binding to the angiotensin converting enzyme 2 (ACE2) viral receptor. This high-throughput method matches the performance of the gold standard live virus infectious assay, as verified with a panel of 206 seropositive donors with varying degrees of infection severity and virus-specific IgG titers, achieving 96.7% sensitivity and 100% specificity. Furthermore, it allows for the parallel assessment of neutralizing activities against multiple SARS-CoV-2 Spike variants of concern (VOC), which is otherwise unpredictable even in individuals displaying robust neutralizing antibody responses. Profiling serum samples from 59 hospitalized COVID-19 patients, it was found that although most had high activity against the 2019-nCoV Spike and to a lesser extent the B.1.1.7 variant, only 58% could efficiently neutralize a Spike derivative containing mutations present in the B.1.351 variant. The cell-free quantitative neutralization assay disclosed herein has proven its clinical relevance in the large-scale evaluation of effective neutralizing antibody responses to VOC after natural infection and that can be applied to the characterization of vaccine-induced antibody responses and of the potency of human monoclonal antibodies.

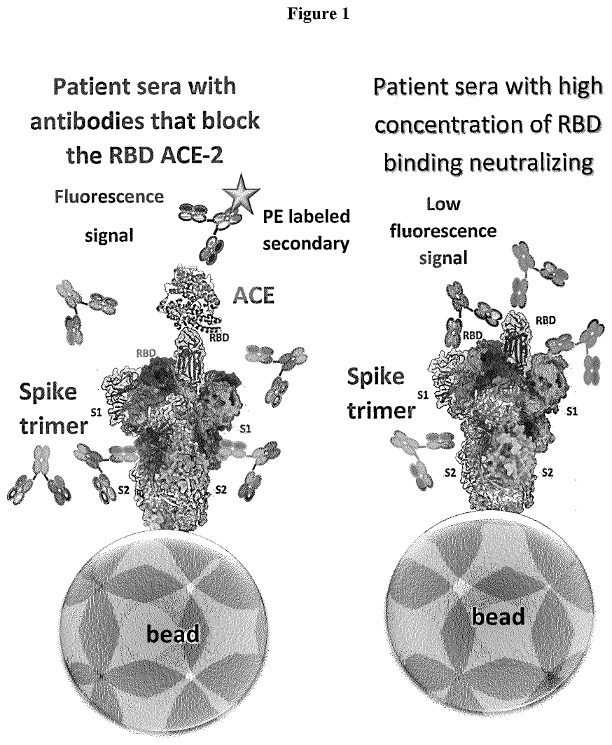

The present innovation provides an in vitro method for the cell-free detection and quantification of virus neutralizing activity, such as the SARS-CoV-2 neutralizing activity, of biological samples (e.g. serum) based on their ability to block the interaction of the prefusion viral protein, such as prefusion trimeric SARS-CoV-2 spike (prefusion SARS-CoV-2 protein), on the one hand and the cell receptor protein, such as angiotensin-converting enzyme 2 (ACE-2) receptor, on the other hand (see FIG. 1).

An aspect of the present invention provides a method for quantifying virus neutralizing antibodies in a biological sample of a subject, wherein the virus is enveloped virus and wherein the method comprises
a) providing at least one prefusion viral protein(s), that is/are stabilized in a prefusion conformation and that is/are reactive with the neutralizing antibodies, and coupling said prefusion viral protein(s) to at least one solid support,
b) obtaining a biological sample from a subject,
c) contacting (mixing) the at least one prefusion viral protein(s) coupled to the at least one solid support with the biological sample to provide a mixture,
d) contacting (adding to) the mixture with one or more cell receptor proteins that bind to the at least one prefusion viral protein(s) or one or more competing antibodies that bind to the at least one prefusion viral protein(s),
e) detecting and quantifying neutralizing antibodies in the biological sample.

In the method of the invention, the virus is enveloped virus, that affects humans or animals, such as
a. RNA viruses:
   Flavivirus (HCV, West Nile virus, dengue virus, tick-borne encephalitis virus, yellow fever virus, Zika virus and several other viruses which may cause encephalitis)
   Alphavirus (e.g. chikungunya, Eastern and Western equine encephalitis virus, etc.)
   Coronavirus, such as SARS-CoV-2
   Hepatitis D
   Orthomyxovirus (flu)
   Paramyxovirus (measles)
   Rhabdovirus (rabies, etc.)
   Bunyavirus (Hanta, Rift Valley, Lassa, Argentine hemorrhagic fever, Crimena-Congo hemorrhagic fever, LCMV)
   Filovirus (Ebola, Marburg)
b. Retroviruses: HIV
c. DNA viruses
   Herpesviruses (HSV, EBV, CMV, etc.)
   Poxviruses
   Hepadnaviruses (HBV)
   Asfarviridae (ASFV)

In one embodiment of the method of the invention, the virus is selected from the group comprising Flavivirus, Alphavirus, Coronavirus, Hepatitis D, Orthomyxovirus, Paramyxovirus, Rhabdovirus, Bunyavirus, Filovirus, Retroviruses, Herpesviruses, Poxviruses, Hepadnaviruses, Asfarviridae. In a preferred embodiment of the method of the invention, the virus is SARS-CoV-2 virus.

Prefusion (envelope) viral proteins from different enveloped viruses listed above and their receptors, when identified, are used in the method of the invention. Examples of such proteins and receptors are provided herein.

The prefusion viral proteins have (soluble) pre-fusion stable state and are stabilized in a multimeric prefusion conformation where critical epitopes are accessible to neutralizing antibodies. The stabilization of the prefusion conformation by the one or more amino acid substitutions, deletions, or insertions can be, for example, energetic stabilization (for example, reducing the energy of the prefusion conformation relative to the post-fusion open conformation) and/or kinetic stabilization (for example, reducing the rate of transition from the otherwise unstable prefusion conformation to the post-fusion conformation). Additionally, stabilization (of for example ectodomain) in the prefusion conformation can include an increase in resistance to denaturation compared to a corresponding native sequence. Accessibility of epitopes of the stabilised viral protein can further be improved by additional chemical or enzymatic treatments of the purified protein. For example, a coronavirus S ectodomain trimer “stabilized in a prefusion conformation” comprises only the ectodomain of the protein, a C-terminal T4 fibritin trimerization motif and two proline mutations to stabilize the metastable prefusion conformation compared to coronavirus S ectodomain trimers formed from a corresponding native coronavirus S sequence.

The prefusion viral protein of the invention, such as coronavirus (SARS-CoV-2) S (spike) protein or fragments thereof can be produced using recombinant techniques, or chemically or enzymatically synthesized; preferably the prefusion viral protein of the invention is produced by recombinant techniques to provide recombinant prefusion viral protein. In some embodiments, the prefusion viral protein of the invention has an amino acid sequence that is not naturally occurring, for example, includes one or more amino acid or nucleic acid substitutions, deletions or insertions.

In the context of the present invention, the prefusion viral proteins are reactive with the neutralizing antibodies means that the prefusion viral proteins comprise one or more epitopes that is recognized by the neutralizing antibodies and that provide binding of the neutralizing antibodies to the prefusion viral proteins. Each type, variant and/or mutant of the prefusion viral proteins can be specific to one or more neutralizing antibodies that are to be quantified.

In the method of the invention, the at least one solid support, to which the at least one prefusion viral protein(s) of the invention is/are coupled, is known in the art and include crosslinked dextran, agarose, polystyrene, polyvinylchloride, cross-linked polyacrylamide, nitrocellulose or nylon-based materials. The solid support to which the prefusion viral protein of the invention is coupled, is selected from the group comprising tubes, membranes, plates, wells of microtiter plates, pads, beads or microspheres; preferably beads or microspheres. The at least one solid support is one or more solid supports. According to an embodiment, each of the at least one solid supports emits a different and distinguishable wavelength after excitation. Typically, each of the at least one prefusion viral protein(s) is coupled to one or more same solid supports. In some other embodiments, each of the at least one prefusion viral protein(s) is coupled to one or more different solid supports. The microspheres or beads can be those available from Luminex Corporation (Austin, TX). Coupling may be to the surface of the microsphere or to an internal surface that is accessible from the outside surface. The method of coupling (attachment) of proteins or antigens to microsphere beads are known in the art. Proteins or antigens can be coupled to beads such as those provided by Luminex Corporation according to the manufacturer's recommendations.

In the method of the invention, the biological sample is a serum sample or a respiratory secretion sample, preferably the biological sample is suspected to contain neutralizing antibodies. The respiratory secretion includes secretions obtained by broncho-alveolar lavage or any other source of antibodies. The biological sample is obtained from a subject that has been either infected by the virus or suspected to have been infected by a virus or that has received a vaccine treatment.

In the method of the invention, the cell receptor protein, which binds to the prefusion viral protein, is a soluble form, such as a soluble form of ACE-2, the main SARS-CoV-2 receptor.

In the method of the invention, the competing antibody that binds to the prefusion viral protein, is typically a known neutralising antibody ideally previously identified with a neutralization assay using viral infection and purified from serum or synthesized.

In the method of the invention, contacting (mixing) the prefusion viral protein coupled to the solid support with the biological sample to provide a mixture is carried out under conditions and during sufficient time, for example about 10 minutes to about 60 minutes, about 20 minutes to 40 minutes, about 30 minutes, to allow binding of the neutralizing antibodies if present in the biological sample, to the prefusion viral protein.

In the method of the invention, contacting (mixing) the at least one prefusion viral protein(s) coupled to the at least one solid support with the biological sample, for example suspected to contain neutralizing antibodies, to provide a mixture, is carried out for time and under conditions sufficient to allow binding of the prefusion viral proteins to the neutralizing antibodies, thereby forming a virial protein/neutralizing antibody complex.

In the method of the invention, contacting (adding to) the mixture with one or more cell receptor proteins that bind to the at least one prefusion viral protein(s) or one or more competing antibodies that bind to the at least one prefusion viral protein(s), is carried out for time and under conditions sufficient to allow binding of the prefusion viral proteins to the cell receptor proteins and/or binding of the prefusion viral proteins to competing antibodies, thereby forming a virial protein/cell receptor complex or viral protein/competing antibody receptor.

In the method of the invention, detecting and quantifying neutralizing antibodies (i.e. measuring neutralizing antibody titer) in the biological sample is carried out by methods known in the art, typically by direct or indirect fluorometric methods (for example with a fluorescence-coupled cell receptor-specific antibody). Alternatively, a modified receptor protein, e.g. directly couple to a fluorescent or chemical tag, can be used, alleviating the need for an anti-receptor antibody or read-out. In some embodiments, quantifying neutralizing antibodies, i.e. measuring neutralizing antibody titer, is done indirectly: the higher the titer, the less receptor (or the previously characterized competing antibody) will bind. In some embodiments, the solid support produces a detectable signal that is detected and thereby allows detection and quantification of neutralizing antibodies.

In the context of SARS-CoV-2 enveloped virus, the viral envelope is typically made up of three proteins that include the membrane protein (M), the envelope protein (E), and the spike protein (S). As compared to the M and E proteins that are primarily involved in virus assembly, the S protein plays a crucial role in penetrating host cells and initiating infection. One of the key biological characteristics of SARS-CoV-2, as well as several other viruses, is the presence of spike proteins that allow these viruses to penetrate host cells and cause infection. The S protein is a highly glycosylated and large type I transmembrane fusion protein that is made up of 1,160 to 1,400 amino acids, depending upon the type of virus. In addition to its role in penetrating cells, the S protein of viruses, particularly the SARS-CoV-2 virus, is a major inducer of neutralizing antibodies. Coronavirus S (spike) protein is initially synthesized as a precursor protein. Individual precursor S polypeptides form a homotrimer and undergo glycosylation within the Golgi apparatus as well as processing to remove the signal peptide, and cleavage by a cellular protease to generate separate S1 and S2 polypeptide chains, which remain associated as S1/S2 protomers within the homotrimer and is therefore a trimer of heterodimers. The S1 subunit is distal to the virus membrane and contains the receptor-binding domain (RBD) that mediates virus attachment to its host (cell) receptor. The S2 subunit contains fusion protein machinery, such as the fusion peptide, two heptad-repeat sequences (HR1 and HR2) and a central helix typical of fusion glycoproteins, a transmembrane domain, and the cytosolic tail domain. A structural conformation adopted by the ectodomain of the coronavirus S protein following processing into a mature coronavirus S protein in the secretory system, and prior to triggering of the fusogenic event that leads to transition of coronavirus S to the postfusion conformation. A coronavirus S ectodomain trimer “stabilized in a prefusion conformation” comprises only the ectodomain of the protein, a C-terminal T4 fibritin trimerization motif and two proline mutations to stabilize the metastable prefusion conformation compared to coronavirus S ectodomain trimers formed from a corresponding native coronavirus S sequence.

The stabilized prefusion ectodomain SARS-CoV-2 protein is provided as SEQ ID NO 1:

MFVFLVLLPLVSSQCVNLTTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YLYRLFRKSNLKPFERDISTEIYQAGSTPCNGVEGENCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCL
IGAEHVNNSYECDIPIGAGICASYQTQTNSPGSASSVASQSIIAYTMSLG
AENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLI
CAQKENGLTVLPPLLTDEMIAQYTSALLAGTITSGWTFGAGAALQIPFAM
QMAYRENGIGVTQNVLYENQKLIANQENSAIGKIQDSLSSTASALGKLQD
VVNQNAQALNTLVKQLSSNFGAISSVLNDILSRLDPPEAEVQIDRLITGR
LQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDL
QELGKYEQ

The stabilized prefusion ectodomain SARS-CoV-2 protein variant alpha is provided as SEQ ID NO 30:

MFVFLVLLPLVSSQCVNLTTRTQLPPAYTNSFTRGVYYPDKVFRSSVLHS
TQDLFLPFFSNVTWFHAISGTNGTKRFDNPVLPFNDGVYFASTEKSNIIR
GWIFGTTLDSKTQSLLIVNNATNVVIKVCEFQFCNDPFLGVYHKNNKSWM
ESEFRVYSSANNCTFEYVSQPFLMDLEGKQGNFKNLREFVFKNIDGYFKI
YSKHTPINLVRDLPQGFSALEPLVDLPIGINITRFQTLLALHRSYLTPGD
SSSGWTAGAAAYYVGYLQPRTFLLKYNENGTITDAVDCALDPLSETKCTL
KSFTVEKGIYQTSNFRVQPTESIVRFPNITNLCPFGEVENATRFASVYAW
NRKRISNCVADYSVLYNSASFSTFKCYGVSPTKLNDLCFTNVYADSFVIR
GDEVRQIAPGQTGKIADYNYKLPDDFTGCVIAWNSNNLDSKVGGNYNYLY
RLFRKSNLKPFERDISTEIYQAGSTPCNGVEGENCYFPLQSYGFQPTYGV
GYQPYRVVVLSFELLHAPATVCGPKKSTNLVKNKCVNFNFNGLTGTGVLT
ESNKKFLPFQQFGRDIDDTTDAVRDPQTLEILDITPCSFGGVSVITPGTN
TSNQVAVLYQGVNCTEVPVAIHADQLTPTWRVYSTGSNVFQTRAGCLIGA
EHVNNSYECDIPIGAGICASYQTQTNSHGSASSVASQSIIAYTMSLGAEN
SVAYSNNSIAIPINFTISVTTEILPVSMTKTSVDCTMYICGDSTECSNLL
LQYGSFCTQLNRALTGIAVEQDKNTQEVFAQVKQIYKTPPIKDFGGFNFS
QILPDPSKPSKRSFIEDLLFNKVTLADAGFIKQYGDCLGDIAARDLICAQ
KFNGLTVLPPLLTDEMIAQYTSALLAGTITSGWTFGAGAALQIPFAMQMA
YRENGIGVTQNVLYENQKLIANQFNSAIGKIQDSLSSTASALGKLQDVVN
QNAQALNTLVKQLSSNFGAISSVLNDILARLDPPEAEVQIDRLITGRLQS
LQTYVTQQLIRAAEIRASANLAATKMSECVLGQSKRVDFCGKGYHLMSFP
QSAPHGVVFLHVTYVPAQEKNFTTAPAICHDGKAHFPREGVFVSNGTHWF
VTQRNFYEPQIITTHNTFVSGNCDVVIGIVNNTVYDPLQPELDSFKEELD
KYFKNHTSPDVDLGDISGINASVVNIQKEIDRLNEVAKNLNESLIDLQEL
GKYEQGSGYIPEAPRDGQAYVRKDGEWVLLSTFLGTSLEVLFQGPGHHHH
HHHHHHSAWSHPQFEKGGGSGGGGSGGSAWSHPQFEK

The stabilized prefusion ectodomain SARS-CoV-2 protein variant beta is provided as SEQ ID NO 31:

MFVFLVLLPLVSSQCVNFTTRTQLPPAYTNSFTRGVYYPDKVFRSSVLHS
TQDLFLPFFSNVTWFHAIHVSGTNGTKRFANPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRGLPQGFSALEPLVDLPIGINITRFQTLHISYLTPGD
SSSGWTAGAAAYYVGYLQPRTFLLKYNENGTITDAVDCALDPLSETKCTL
KSFTVEKGIYQTSNFRVQPTESIVRFPNITNLCPFGEVFNATRFASVYAW
NRKRISNCVADYSVLYNSASFSTFKCYGVSPTKLNDLCFTNVYADSFVIR
GDEVRQIAPGQTGNIADYNYKLPDDFTGCVIAWNSNNLDSKVGGNYNYLY
RLFRKSNLKPFERDISTEIYQAGSTPCNGVKGFNCYFPLQSYGFQPTYGV
GYQPYRVVVLSFELLHAPATVCGPKKSTNLVKNKCVNFNFNGLTGTGVLT
ESNKKFLPFQQFGRDIADTTDAVRDPQTLEILDITPCSFGGVSVITPGTN
TSNQVAVLYQGVNCTEVPVAIHADQLTPTWRVYSTGSNVFQTRAGCLIGA
EHVNNSYECDIPIGAGICASYQTQTNSPGSASSVASQSIIAYTMSLGVEN
SVAYSNNSIAIPTNFTISVTTEILPVSMTKTSVDCTMYICGDSTECSNLL
LQYGSFCTQLNRALTGIAVEQDKNTQEVFAQVKQIYKTPPIKDFGGFNFS
QILPDPSKPSKRSFIEDLLFNKVTLADAGFIKQYGDCLGDIAARDLICAQ
KFNGLTVLPPLLTDEMIAQYTSALLAGTITSGWTFGAGAALQIPFAMQMA
YRENGIGVTQNVLYENQKLIANQFNSAIGKIQDSLSSTASALGKLQDVVN
QNAQALNTLVKQLSSNFGAISSVLNDILSRLDPPEAEVQIDRLITGRLQS
LQTYVTQQLIRAAEIRASANLAATKMSECVLGQSKRVDFCGKGYHLMSFP
QSAPHGVVFLHVTYVPAQEKNFTTAPAICHDGKAHFPREGVFVSNGTHWF
VTQRNFYEPQIITTDNTFVSGNCDVVIGIVNNTVYDPLQPELDSFKEELD
KYFKNHTSPDVDLGDISGINASVVNIQKEIDRLNEVAKNLNESLIDLQEL
GKYEQGSGYIPEAPRDGQAYVRKDGEWVLLSTFLGRSLEVLFQGPGHHHH
HHHHSAWSHPQFEKGGGSGGGGSGGSAWSHPQFEK

The stabilized prefusion ectodomain SARS-CoV-2 protein variant delta is provided as SEQ ID NO 32:

MFVFLVLLPLVSSQCVNLRTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESGVYSSANNCTFEYVSQPFLMDLEGKQGNFKNLREFVFKNIDGYFK
IYSKHTPINLVRDLPQGFSALEPLVDLPIGINITRFQTLLALHRSYLTPG
DSSSGWTAGAAAYYVGYLQPRTFLLKYNENGTITDAVDCALDPLSETKCT
LKSFTVEKGIYQTSNFRVQPTESIVRFPNITNLCPFGEVFNATRFASVYA
WNRKRISNCVADYSVLYNSASFSTFKCYGVSPTKLNDLCFTNVYADSFVI
RGDEVRQIAPGQTGKIADYNYKLPDDFTGCVIAWNSNNLDSKVGGNYNYR
YRLFRKSNLKPFERDISTEIYQAGSKPCNGVEGENCYFPLQSYGFQPTNG
VGYQPYRVVVLSFELLHAPATVCGPKKSTNLVKNKCVNFNFNGLTGTGVL
TESNKKFLPFQQFGRDIADTTDAVRDPQTLEILDITPCSFGGVSVITPGT
NTSNQVAVLYQGVNCTEVPVAIHADQLTPTWRVYSTGSNVFQTRAGCLIG
AEHVNNSYECDIPIGAGICASYQTQTNSRGSASSVASQSIIAYTMSLGAE
NSVAYSNNSIAIPTNFTISVTTEILPVSMTKTSVDCTMYICGDSTECSNL
LLQYGSFCTQLNRALTGIAVEQDKNTQEVFAQVKQIYKTPPIKDFGGFNF
SQILPDPSKPSKRSFIEDLLFNKVTLADAGFIKQYGDCLGDIAARDLICA
QKFNGLTVLPPLLTDEMIAQYTSALLAGTITSGWTFGAGAALQIPFAMQM
AYRENGIGVTQNVLYENQKLIANQFNSAIGKIQDSLSSTASALGKLQNVV
NQNAQALNTLVKQLSSNFGAISSVLNDILSRLDPPEAEVQIDRLITGRLQ
SLQTYVTQQLIRAAEIRASANLAATKMSECVLGQSKRVDFCGKGYHLMSF
PQSAPHGVVFLHVTYVPAQEKNFTTAPAICHDGKAHFPREGVFVSNGTHW
FVTQRNFYEPQIITTDNTFVSGNCDVVIGIVNNTVYDPLQPELDSFKEEL
DKYFKNHTSPDVDLGDISGINASVVNIQKEIDRLNEVAKNLNESLIDLQE
LGKYEQGSGYIPEAPRDGQAYVRKDGEWVLLSTFLGRSLEVLFQGPGHHH
HHHHHSAWSHPQFEKGGGSGGGGSGGSAWSHPQFEK

The stabilized prefusion ectodomain SARS-CoV-2 protein variant gamma is provided as SEQ ID NO 33:

MFVFLVLLPLVSSQCVNFTNRTQLPSAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNYPFLGVYYHKNNK
SWMESEFRVYSSANNCTFEYVSQPFLMDLEGKQGNFKNLSEFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSF
VIRGDEVRQIAPGQTGTIADYNYKLPDDFTGCVIAWNSNNLDSKVGGNYN
YLYRLFRKSNLKPFERDISTEIYQAGSTPCNGVKGFNCYFPLQSYGFQPT
YGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQGVNCTEVPVAIHADQLTPTWRVYSTGSNVFQTRAGCL
IGAEYVNNSYECDIPIGAGICASYQTQTNSPGSASSVASQSIIAYTMSLG
AENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAM
QMAYRENGIGVTQNVLYENQKLIANQFNSAIGKIQDSLSSTASALGKLQD
VVNQNAQALNTLVKQLSSNFGAISSVLNDILSRLDPPEAEVQIDRLITGR
LQSLQTYVTQQLIRAAEIRASANLAAIKMSECVLGQSKRVDFCGKGYHLM
SFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKE
ELDKYFKNHTSPDVDLGDISGINASFVNIQKEIDRLNEVAKNLNESLIDL
QELGKYEQGSGYIPEAPRDGQAYVRKDGEWVLLSTFLGRSLEVLFQGPGH
HHHHHHHHHSAWSHPQFEKGGGSGGGGSGGSAWSHPQFEK

The stabilized prefusion ectodomain SARS-CoV-2 protein variant kappa is provided as SEQ ID NO 34:

MFVFLVLLPLVSSQCVNLTTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMKSEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVENATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YRYRLFRKSNLKPFERDISTEIYQAGSTPCNGVQGFNCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQGVNCTEVPVAIHADQLTPTWRVYSTGSNVFQTRAGCL
IGAEHVNNSYECDIPIGAGICASYQTQTNSRGSASSVASQSIIAYTMSLG
AENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQD
VVNQNAQALNTLVKQLSSNFGAISSVLNDILSRLDPPEAEVQIDRLITGR
LQSLQTYVTQQLIRAAEIRASANLAATKMSECVLGQSKRVDFCGKGYHLM
SFPQSAPHGVVFLHVTYVPAHEKNFTTAPAICHDGKAHFPREGVFVSNGT
HWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDL
QELGKYEQGSGYIPEAPRDGQAYVRKDGEWVLLSTFLGRSLEVLFQGPGH
HHHHHHSAWSHPQFEKGGGSGGGGSGGSAWSHPQFEK

The stabilized prefusion ectodomain SARS-CoV-2 protein variant lambda is provided as SEQ ID NO 35:

MFVFLVLLPLVSSQCVNLTTRTQLPPAYTNSFTRGVYYPDKVFRSSVLH
STQDLFLPFFSNVTWFHAIHVSGTNVIKRFDNPVLPFNDGVYFASTEKS
NIIRGWIFGTTLDSKTQSLLIVNNATNVVIKVCEFQFCNDPFLGVYYHK
NNKSWMESEFRVYSSANNCTFEYVSQPFLMDLEGKQGNFKNLREFVFKN
IDGYFKIYSKHTPINLVRDLPQGFSALEPLVDLPIGINITRFQTLLALH
NSSSGWTAGAAAYYVGYLQPRTFLLKYNENGTITDAVDCALDPLSETKC
TLKSFTVEKGIYQTSNFRVQPTESIVRFPNITNLCPFGEVENATRFASV
YAWNRKRISNCVADYSVLYNSASFSTFKCYGVSPTKLNDLCFTNVYADS
FVIRGDEVRQIAPGQTGKIADYNYKLPDDFTGCVIAWNSNNLDSKVGGN
YNYQYRLFRKSNLKPFERDISTEIYQAGSTPCNGVEGFNCYSPLQSYGF
QPTNGVGYQPYRVVVLSFELLHAPATVCGPKKSTNLVKNKCVNFNFNGL
TGTGVLTESNKKFLPFQQFGRDIADTTDAVRDPQTLEILDITPCSFGGV
SVITPGTNTSNQVAVLYQGVNCTEVPVAIHADQLTPTWRVYSTGSNVFQ
TRAGCLIGAEHVNNSYECDIPIGAGICASYQTQTNSPGSASSVASQSII
AYTMSLGAENSVAYSNNSIAIPTNFTISVTTEILPVSMTKTSVDCTMYI
CGDSTECSNLLLQYGSFCTQLNRALTGIAVEQDKNTQEVFAQVKQIYKT
PPIKDFGGFNFSQILPDPSKPSKRSFIEDLLFNKVTLADAGFIKQYGDC
LGDIAARDLICAQKFNGLNVLPPLLTDEMIAQYTSALLAGTITSGWTFG
AGAALQIPFAMQMAYRENGIGVTQNVLYENQKLIANQFNSAIGKIQDSL
SSTASALGKLQDVVNQNAQALNTLVKQLSSNFGAISSVLNDILSRLDPP
EAEVQIDRLITGRLQSLQTYVTQQLIRAAEIRASANLAATKMSECVLGQ
SKRVDFCGKGYHLMSFPQSAPHGVVFLHVTYVPAQEKNFTTAPAICHDG
KAHFPREGVFVSNGTHWFVTQRNFYEPQIITTDNTFVSGNCDVVIGIVN
NTVYDPLQPELDSFKEELDKYFKNHTSPDVDLGDISGINASVVNIQKEI
DRLNEVAKNLNESLIDLQELGKYEQGSGYIPEAPRDGQAYVRKDGEWVL
LSTFLGRSLEVLFQGPGHHHHHHHHSAWSHPQFEKGGGSGGGGSGGSAW
SHPQFEK

The stabilized prefusion ectodomain SARS-CoV-2 protein variant mu is provided as SEQ ID NO 36:

MFVFLVLLPLVSSQCVNLTTRTQLPPAYTNSFTRGVYYPDKVFRSSVLH
STQDLFLPFFSNVTWFHAIHVSGTNGTKRFDNPVLPFNDGVYFASIEKS
NIIRGWIFGTTLDSKTQSLLIVNNATNVVIKVCEFQFCNDPFLGVTSNH
KNNKSWMESEFRVYSSANNCTFEYVSQPFLMDLEGKQGNFKNLREFVFK
NIDGYFKIYSKHTPINLVRDLPQGFSALEPLVDLPIGINITRFQTLLAL
HRSYLTPGDSSSGWTAGAAAYYVGYLQPRTFLLKYNENGTITDAVDCAL
DPLSETKCTLKSFTVEKGIYQTSNFRVQPTESIVRFPNITNLCPFGEVF
NATKFASVYAWNRKRISNCVADYSVLYNSASFSTFKCYGVSPTKLNDLC
FTNVYADSFVIRGDEVRQIAPGQTGKIADYNYKLPDDFTGCVIAWNSNN
LDSKVGGNYNYLYRLFRKSNLKPFERDISTEIYQAGSTPCNGVKGFNCY
FPLQSYGFQPTYGVGYQPYRVVVLSFELLHAPATVCGPKKSTNLVKNKC
VNFNFNGLTGTGVLTESNKKFLPFQQFGRDIADTTDAVRDPQTLEILDI
TPCSFGGVSVITPGTNTSNQVAVLYQGVNCTEVPVAIHADQLTPTWRVY
STGSNVFQTRAGCLIGAEHVNNSYECDIPIGAGICASYQTQTNSHGSAS
SVASQSIIAYTMSLGAENSVAYSNNSIAIPTNFTISVTTEILPVSMTKT
SVDCTMYICGDSTECSNLLLQYGSFCTQLNRALTGIAVEQDKNTQEVFA
QVKQIYKTPPIKDFGGFNFSQILPDPSKPSKRSFIEDLLFNKVTLADAG
FIKQYGDCLGDIAARDLICAQKFNGLTVLPPLLTDEMIAQYTSALLAGT
ITSGWTFGAGAALQIPFAMQMAYRENGIGVTQNVLYENQKLIANQFNSA
IGKIQDSLSSTASALGKLQNVVNQNAQALNTLVKQLSSNFGAISSVLND
ILSRLDPPEAEVQIDRLITGRLQSLQTYVTQQLIRAAEIRASANLAATK
MSECVLGQSKRVDFCGKGYHLMSFPQSAPHGVVFLHVTYVPAQEKNFTT
APAICHDGKAHFPREGVFVSNGTHWFVTQRNFYEPQIITTDNTFVSGNC
DVVIGIVNNTVYDPLQPELDSFKEELDKYFKNHTSPDVDLGDISGINAS
VVNIQKEIDRLNEVAKNLNESLIDLQELGKYEQGSGYIPEAPRDGQAYV
RKDGEWVLLSTFLGRSLEVLFQGPGHHHHHHHHSAWSHPQFEKGGGSGG
GGSGGSAWSHPQFEK

The cell receptor ACE2 (ACE-2-Fc fusion protein) is provided as SEQ ID NO 2:

STIEEQAKTFLDKFNHEAEDLFYQSSLASWNYNTNITEENVQNMNNAGD
KWSAFLKEQSTLAQMYPLQEIQNLTVKLQLQALQQNGSSVLSEDKSKRL
NTILNTMSTIYSTGKVCNPDNPQECLLLEPGLNEIMANSLDYNERLWAW
ESWRSEVGKQLRPLYEEYVVLKNEMARANHYEDYGDYWRGDYEVNGVDG
YDYSRGQLIEDVEHTFEEIKPLYEHLHAYVRAKLMNAYPSYISPIGCLP
AHLLGDMWGRFWTNLYSLTVPFGQKPNIDVTDAMVDQAWDAQRIFKEAE
KFFVSVGLPNMTQGFWENSMLTDPGNVQKAVCHPTAWDLGKGDFRILMC
TKVTMDDFLTAHHEMGHIQYDMAYAAQPFLLRNGANEGFHEAVGEIMSL
SAATPKHLKSIGLLSPDFQEDNETEINFLLKQALTIVGTLPFTYMLEKW
RWMVFKGEIPKDQWMKKWWEMKREIVGVVEPVPHDETYCDPASLFHVSN
DYSFIRYYTRTLYQFQFQEALCQAAKHEGPLHKCDISNSTEAGQKLFNM
LRLGKSEPWTLALENVVGAKNMNVRPLLNYFEPLFTWLKDQNKNSFVGW
STDWSPYADGSGLEVLFQGPGSAWSHPQFEKGGGSGGGSGGSAWSHPQ
FEKGSHHHHHHHHHH

The stabilized ENV prefusion ectodomain of the HIV retrovirus is provided as SEQ ID NO 3:

AENLWVTVYYGVPVWKDAETTLFCASDAKAYETEKHNVWATHCCVPTDP
NPQEIHLENVTEEFNMWKNNMVEQMHTDIISLWDQSLKPCVKLTPLCVT
LQCTNVTNNITDDMRGELKNCSFNMTTELRDKKQKVYSLFYRLDVVQIN
ENQGNRSNNSNKEYRLINCNTSAITQACPKVSFEPIPIHYCAPAGFAIL
KCKDKKFNGTGPCPSVSTVQCTHGIKPVVSTQLLLNGSLAEEEVMIRSE
NITNNAKNILVQFNTPVQINCTRPNNNTRKSIRIGPGQWFYATGDIIGD
IRQAHCNVSKATWNETLGKVVKQLRKHFGNNTIIRFANSSGGDLEVTTH
SFNCGGEFFYCNTSGLFNSTWISNTSVQGSNSTGSNDSITLPCRIKQII
NMWQRIGQAMYAPPIQGVIRCVSNITGLILTRDGGSTNSTTETFRPGGG
DMRDNWRSELYKYKVVKIEPLGVAPTRCKRRVVGRRRRRRAVGIGAVFL
GFLGAAGSTMGAASMTLTVQARNLLSGIVQQQSNLLRAPECQQHLLKLT
VWGIKQLQARVLAVERYLRDQQLLGIWGCSGKLICCTNVPWNSSWSNRN
LSEIWDNMTWLQWDKEISNYTQIIYGLLEESQNQQEKNEQDLLALD

The HIV human CD4 T-cell receptor is provided as SEQ ID NO 4:

MNRGVPFRHLLLVLQLALLPAATQGKKVVLGKKGDTVELTCTASQKKSI
QFHWKNSNQIKILGNQGSFLTKGPSKLNDRADSRRSLWDQGNFPLIIKN
LKIEDSDTYICEVEDQKEEVQLLVFGLTANSDTHLLQGQSLTLTLESPP
GSSPSVQCRSPRGKNIQGGKTLSVSQLELQDSGTWTCTVLQNQKKVEFK
IDIVVLAFQKASSIVYKKEGEQVEFSFPLAFTVEKLTGSGELWWQAERA
SSSKSWITFDLKNKEVSVKRVTQDPKLQMGKKLPLHLTLPQALPQYAGS
GNLTLALEAKTGKLHQEVNLVVMRATQLQKNLTCEVWGPTSPKLMLSLK
LENKEAKVSKREKAVWVLNPEAGMWQCLLSDSGQVLLESNIKVLPTWST
PVQPMALIVLGGVAGLLLFIGLGIFFCVRCRHRRRQAERMSQIKRLLSE
KKTCQCPHRFQKTCSPI

The stabilized GP prefusion ectodomain of the Ebola (EBOV) filovirus is provided as SEQ ID NO 5:

ETGRSIPLGVIHNSALQVSDVDKLVCRDKLSSTNQLRSVGLNLEGNGVA
TDVPSATKRWGFRSGVPPKVVNYEAGEWAENCYNLEIKKPDGSECLPAA
PDGIRGFPRCRYVHKVSGTGPCAGDFAFHKEGAFFLYDRLASTVIYRGT
TFAEGVVAFLILPQAKKDFFSSHPLREPVNATEDPSSGYYSTTIRYQAT
GFGTNETEYLFEVDNLTYVQLESRFTPQFLLQLNETIYTSGKRSNTTGK
LIWKVNPEIDTTIGEWAFWETKKNLTRKIRSEELSFTVVSTHHQDTGEE
SASSGKLGLITNTIAGVAGLITGGRRTRRXXXXXXXXXEAIVNAQPKCN
PNLHYWTTQDEGAAIGLAWIPYFGPAAEGIYIEGLMHNQDGLICGLRQL
ANETTQALQLFLRATTELRTFSILNRKAIDFLLQRWGGTCHILGPDCCI
EPHDWTKNITDKIDQIIHDFVDGSGYIPEAPRDGQAYVRKDGEWVLLST
FLGTHHHHHH

The Ebola human cholesterol transporter 1 is provided as SEQ ID NO 6:

MTARGLALGLLLLLLCPAQVFSQSCVWYGECGIAYGDKRYNCEYSGPPK
PLPKDGYDLVQELCPGFFFGNVSLCCDVRQLQTLKDNLQLPLQFLSRCP
SCFYNLLNLFCELTCSPRQSQFLNVTATEDYVDPVTNQTKTNVKELQYY
VGQSFANAMYNACRDVEAPSSNDKALGLLCGKDADACNATNWIEYMFNK
DNGQAPFTITPVFSDFPVHGMEPMNNATKGCDESVDEVTAPCSCQDCSI
VCGPKPQPPPPPAPWTILGLDAMYVIMWITYMAFLLVFFGAFFAVWCYR
KRYFVSEYTPIDSNIAFSVNASDKGEASCCDPVSAAFEGCLRRLFTRWG
SFCVRNPGCVIFFSLVFITACSSGLVFVRVTTNPVDLWSAPSSQARLEK
EYFDQHFGPFFRTEQLIIRAPLTDKHIYQPYPSGADVPFGPPLDIQILH
QVLDLQIAIENITASYDNETVTLQDICLAPLSPYNTNCTILSVLNYFQN
SHSVLDHKKGDDFFVYADYHTHFLYCVRAPASLNDTSLLHDPCLGTFGG
PVFPWLVLGGYDDQNYNNATALVITFPVNNYYNDTEKLQRAQAWEKEFI
NFVKNYKNPNLTISFTAERSIEDELNRESDSDVFTVVISYAIMFLYISL
ALGHMKSCRRLLVDSKVSLGIAGILIVLSSVACSLGVFSYIGLPLTLIV
IEVIPFLVLAVGVDNIFILVQAYQRDERLQGETLDQQLGRVLGEVAPSM
FLSSFSETVAFFLGALSVMPAVHTFSLFAGLAVFIDFLLQITCFVSLLG
LDIKRQEKNRLDIFCCVRGAEDGTSVQASESCLFRFFKNSYSPLLLKDW
MRPIVIAIFVGVLSFSIAVLNKVDIGLDQSLSMPDDSYMVDYFKSISQY
LHAGPPVYFVLEEGHDYTSSKGQNMVCGGMGCNNDSLVQQIFNAAQLDN
YTRIGFAPSSWIDDYFDWVKPQSSCCRVDNITDQFCNASVVDPACVRCR
PLTPEGKQRPQGGDFMRFLPMFLSDNPNPKCGKGGHAAYSSAVNILLGH
GTRVGATYFMTYHTVLQTSADFIDALKKARLIASNVTETMGINGSAYRV
FPYSVFYVFYEQYLTIIDDTIFNLGVSLGAIFLVTMVLLGCELWSAVIM
CATIAMVLVNMFGVMWLWGISLNAVSLVNLVMSCGISVEFCSHITRAFT
VSMKGSRVERAEEALAHMGSSVFSGITLTKFGGIVVLAFAKSQIFQIFY
FRMYLAMVLLGATHGLIFLPVLLSYIGPSVNKAKSCATEERYKGTERER
LLNF

The prefusion F protein of the RSV paramyxovirus is provided as SEQ ID NO 7:

QNITEEFYQSTCSAVSKGYLSALRTGWYTSVITIELSNIKKIKCNGTDA
KIKLIKQELDKYKNAVTELQLLMQSTPATNNQARGSGSGRSLGFLLGVG
SAIASGVAVSKVLHLEGEVNKIKSALLSTNKAVVSLSNGVSVLTSKVLD
LKNYIDKQLLPIVNKQSCSIPNIETVIEFQQKNNRLLEITREFSVNAGV
TTPVSTYMLTNSELLSLINDMPITNDQKKLMSNNVQIVRQQSYSIMSII
KEEVLAYVVQLPLYGVIDTPCWKLHTSPLCTTNTKEGSNICLTRTDRGW
YCDNAGSVSFFPQAETCKVQSNRVFCDTMNSLTLPSEVNLCNVDIFNPK
YDCKIMTSKTDVSSSVITSLGAIVSCYGKTKCTASNKNRGIIKTFSNGC
DYVSNKGVDTVSVGNTLYYVNKQEGKSLYVKGEPIINFYDPLVFPSDQF
DASISQVNEKINQSLAFIRKSDELLSAIGGYIPEAPRDGQAYVRKDGEW
VLLSTFL

The RSV human chemokine receptor 1 receptor is provided as SEQ ID NO 8:

MDQFPESVTENFEYDDLAEACYIGDIVVFGTVFLSIFYSVIFAIGLVGN
LLVVFALTNSKKPKSVTDIYLLNLALSDLLFVATLPFWTHYLINEKGLH
NAMCKFTTAFFFIGFFGSIFFITVISIDRYLAIVLAANSMNNRTVQHGV
TISLGVWAAAILVAAPQFMFTKQKENECLGDYPEVLQEIWPVLRNVETN
FLGFLLPLLIMSYCYFRIIQTLFSCKNHKKAKAIKLILLVVIVFFLFWT
PYNVMIFLETLKLYDFFPSCDMRKDLRLALSVTETVAFSHCCLNPLIYA
FAGEKFRRYLYHLYGKCLAVLCGRSVHVDFSSSESQRSRHGSVLSSNFT
YHTSDGDALLLL

The prefusion F protein of the Nipah virus paramyxovirus is provided as SEQ ID NO 9:

MVVILDKRCYCNLLILILMISECSVGILHYEKLSKIGLVKGVTRKYKIK
SNPLTKDIVIKMIPNVSDMSQCTGSVMENYKTRLNGILTPIKGALEIYK
NNTHDLVGDVRLAGVIMAGVAIGIATAAQITAGVALYEAMKNADNINKL
KSSIESTNEAVVKLQETAEKTVYVLTALQDYINTNLVPTIDKISCKQTE
LSLDLALSKYLSDLLFVFGPNLQDPVSNSMTIQAISQAFGGNYETLLRT
LGYATEDFDDLLESDSITGQIIYVDLSSYYIIVRVYFPILTEIQQAYIQ
ELLPVSFNNDDSEWISIVPNFILVRNTLISNIEIGFCLITKRSVICNQD
YATPMTNNMRECLTGSTEKCPRELVVSSHVPRFALSNGVLFANCISVTC
QCQTTGRAISQSGEQTLLMIDNTTCPTAVLGNVIISLGKYLGSVNYNSE
GIAIGPPVFTDKVDISSQISSMNQSLQQSKDYIKEAQRLLDTVNPSLKL
MKQIEDKIEEILSKIYHIENEIARIKKLIGEAPGGIEGR

The Nipah virus EphrinB2 receptor is provided as SEQ ID NO 10:

MAVRRDSVWKYCWGVLMVLCRTAISKSIVLEPIYWNSSNSKFLPGQGLV
LYPQIGDKLDIICPKVDSKTVGQYEYYKVYMVDKDQADRCTIKKENTPL
LNCAKPDQDIKFTIKFQEFSPNLWGLEFQKNKDYYIISTSNGSLEGLDN
QEGGVCQTRAMKILMKVGQDASSAGSTRNKDPTRRPELEAGTNGRSSTT
SPFVKPNPGSSTDGNSAGHSGNNILGSEVALFAGIASGCIIFIVIIITL
VVLLLKYRRRHRKHSPQHTTTLSLSTLATPKRSGNNNGSEPSDIIIPLR
TADSVFCPHYEKVSGDYGHPVYIVQEMPPQSPANIYYKV

The prefusion E protein of the Yellow Fever virus (YFV) Flavivirus is provided as SEQ ID NO 11:

AHCIGITDRDFIEGVHGGTWVSATLEQDKCVTVMAPDKPSLDISLETVA
IDRPAEVRKVCYNAVLTHVKINDKCPSTGEAHLAEENEGDNACKRTYSD
RGWGNGCGLFGKGSIVACAKFTCAKSMSLFEVDQTKIQYVIRAQLHVGA
KQENWNTDIKTLKFDALSGSQEVEFIGYGKATLECQVQTAVDFGNSYIA
EMETESWIVDRQWAQDLTLPWQSGSGGVWREMHHLVEFEPPHAATIRVL
ALGNQEGSLKTALTGAMRVTKDTNDNNLYKLHGGHVSCRVKLSALTLKG
TSYKICTDKMFFVKNPTDTGHGTVVMQVKVSKGAPCRIPVIVADDLTAA
INKGILVTVNPIASTNDDEVLIEVNPPFGDSYIIVGRGDSRLTYQWHKE
GSS

The E protein ectodomain of the Zika Flavivirus is provided as SEQ ID NO 12:

MIRCIGVSNRDFVEGMSGGTWVDVVLEHGGCVTVMAQDKPTVDIELVTT
TVSNMAEVRSYCYEASISDMASDSRCPTQGEAYLDKQSDTQYVCKRTLV
DRGWGNGCGLFGKGSLVTCAKFACSKKMTGKSIQPENLEYRIMLSVHGS
QHSGMIVNDTGHETDENRAKVEITPNSPRAEATLGGFGSLGLDCEPRTG
LDFSDLYYLTMNNKHWLVHKEWFHDIPLPWHAGADTGTPHWNNKEALVE
FKDAHAKRQTVVVLGSQEGAVHTALAGALEAEMDGAKGRLSSGHLKCRL
KMDKLRLKGVSYSLCTAAFTFTKIPAETLHGTVTVEVQYAGTDGPCKVP
AQMAVDMQTLTPVGRLITANPVITESTENSKMMLELDPPFGDSYIVIGV
GEKKITHHWHRSGSTIGKHHHHHH

An example of the Zika virus C-type lectin receptors is provided as SEQ ID NO 13:

MSDSKEPRLQQLGLLEEEQLRGLGFRQTRGYKSLAGCLGHGPLVLQLLS
FTLLAGLLVQVSKVPSSISQEQSRQDAIYQNLTQLKAAVGELSEKSKLQ
EIYQELTQLKAAVGELPEKSKLQEIYQELTRLKAAVGELPEKSKLQEIY
QELTWLKAAVGELPEKSKMQEIYQELTRLKAAVGELPEKSKQQEIYQEL
TRLKAAVGELPEKSKQQEIYQELTRLKAAVGELPEKSKQQEIYQELTQL
KAAVERLCHPCPWEWTFFQGNCYFMSNSQRNWHDSITACKEVGAQLVVI
KSAEEQNFLQLQSSRSNRFTWMGLSDLNQEGTWQWVDGSPLLPSFKQYW
NRGEPNNVGEEDCAEFSGNGWNDDKCNLAKFWICKKSAASCSRDEEQFL
SPAPATPNPPPA

The p62-E1 protein of the Chikungunya Alphavirus is provided as

P62 envelope glycoprotein (SEQ ID NO 14)

PVMCLLANTTFPCSQPPCTPCCYEKEPEETLRMLEDNVMRPGYYQLLQA
SLTCSPHRQRESTKDNFNVYKATRPYLAHCPDCGEGHSCHSPVALERIR
NEATDGTLKIQVSLQIGIKTDDSHDWTKLRYMDNHMPADAERAGLFVRT
SAPCTITGTMGHFILARCPKGETLTVGFTDSRKISHSCTHPFHHDPPVI
GREKFHSRPQHGKELPCSTYVQSTAATTEEIEVHMPPDTPDRTLMSQQS
GNVKITVNGQTVRYKCNCGGSNEGLTTTDKVINNCKVDQCHAAVTNHKK
WQYNSPLVPRNAELGDRKGKIHIPFPLANVTCRVPKARNPTVTYGKNQV
IMLLYPDHPTLLSYRNMGEEPNYQEEWVMHKKEVVLTVPTEGLEVTWGN
NEPYKYWPQ

E1 envelope glycoprotein (SEQ ID NO 15)

GGYEHVTVIPNTVGVPYKTLVNRPGYSPMVLEMELLSVTLEPTLSLDYI
TCEYKTVIPSPYVKCCGTAECKDKNLPDYSCKVFTGVYPFMWGGAYCFC
DAENTQLSEAHVEKSESCKTEFASAYRAHTASASAKLRVLYQGNNITVT
AYANGDHAVTVKDAKFIVGPMSSAWTPFDNKIVVYKGDVYNMDYPPFGA
GRPGQFGDIQSRTPESKDVYANTQLVLQRPAAGTVHVPYSQAPSGFKYW
LKERGASLQHTAPFGCQIATNPVRAVNCAVGNMPISIDIPEAAFTRVVD
APSLTDMSCEVPACTHSSDFGGVAIIKYAASKKGKCAVHSMTNAVTIRE
AEIEVEGNSQLQISFSTALASAEFRVQVCSTQVHCAAECHPPKDHIVNYP

The Chikungunya Matrix remodeling associated protein 8 (MXRA8) receptor is provided as SEQ ID NO 16:

MALPSRILLWKLVLLQSSAVLLHSGSSVPAAAGSSVVSESAVSWEAGAR
AVLRCQSPRMVWTQDRLHDRQRVLHWDLRGPGGGPARRLLDLYSAGEQR
VYEARDRGRLELSASAFDDGNFSLLIRAVEETDAGLYTCNLHHHYCHLY
ESLAVRLEVTDGPPATPAYWDGEKEVLAVARGAPALLTCVNRGHVWTDR
HVEEAQQVVHWDRQPPGVPHDRADRLLDLYASGERRAYGPLFLRDRVAV
GADAFERGDFSLRIEPLEVADEGTYSCHLHHHYCGLHERRVFHLTVAEP
HAEPPPRGSPGNGSSHSGAPGPDPTLARGHNVINVIVPESRAHFFQQLG
YVLATLLLFILLLVTVLLAARRRRGGYEYSDQKSGKSKGKDVNLAEFAV
AAGDQMLYRSEDIQLDYKNNILKERAELAHSPLPAKYIDLDKGFRKENC
K

The prefusion stabilized F protein of the RSV paramyxovirus is provided as SEQ ID NO 17:

QNITEEFYQSTCSAVSKGYLSALRTGWYTSVITIELSNIKKIKCNGTDA
KIKLIKQELDKYKNAVTELQLLMQSTPATNNQARGSGSGRSLGFLLGVG
SAIASGVAVSKVLHLEGEVNKIKSALLSTNKAVVSLSNGVSVLTSKVLD
LKNYIDKQLLPIVNKQSCSIPNIETVIEFQQKNNRLLEITREFSVNAGV
TTPVSTYMLTNSELLSLINDMPITNDQKKLMSNNVQIVRQQSYSIMSII
KEEVLAYVVQLPLYGVIDTPCWKLHTSPLCTTNTKEGSNICLTRTDRGW
YCDNAGSVSFFPQAETCKVQSNRVFCDTMNSLTLPSEVNLCNVDIFNPK
YDCKIMTSKTDVSSSVITSLGAIVSCYGKTKCTASNKNRGIIKTFSNGC
DYVSNKGVDTVSVGNTLYYVNKQEGKSLYVKGEPIINFYDPLVFPSDQF
DASISQVNEKINQSLAFIRKSDELLSAIGGYIPEAPRDGQAYVRKDGEW
VLLSTFL

The RSV CX3CR1 chemokine receptor is provided as SEO ID NO 18:

MDQFPESVTENFEYDDLAEACYIGDIVVFGTVFLSIFYSVIFAIGLVGN
LLVVFALTNSKKPKSVTDIYLLNLALSDLLFVATLPFWTHYLINEKGLH
NAMCKFTTAFFFIGFFGSIFFITVISIDRYLAIVLAANSMNNRTVQHGV
TISLGVWAAAILVAAPQFMFTKQKENECLGDYPEVLQEIWPVLRNVETN
FLGFLLPLLIMSYCYFRIIQTLFSCKNHKKAKAIKLILLVVIVFFLFWT
PYNVMIFLETLKLYDFFPSCDMRKDLRLALSVTETVAFSHCCLNPLIYA
FAGEKFRRYLYHLYGKCLAVLCGRSVHVDFSSSESQRSRHGSVLSSNFT
YHTSDGDALLLL

The prefusion stabilized F protein of the Nipah virus Paramyxovirus is provided as SEQ ID NO 19:

MVVILDKRCYCNLLILILMISECSVGILHYEKLSKIGLVKGVTRKYKIK
SNPLTKDIVIKMIPNVSDMSQCTGSVMENYKTRLNGILTPIKGALEIYK
NNTHDLVGDVRLAGVIMAGVAIGIATAAQITAGVALYEAMKNADNINKL
KSSIESTNEAVVKLQETAEKTVYVLTALQDYINTNLVPTIDKISCKQTE
LSLDLALSKYLSDLLFVFGPNLQDPVSNSMTIQAISQAFGGNYETLLRT
LGYATEDFDDLLESDSITGQIIYVDLSSYYIIVRVYFPILTEIQQAYIQ
ELLPVSFNNDDSEWISIVPNFILVRNTLISNIEIGFCLITKRSVICNQD
YATPMTNNMRECLTGSTEKCPRELVVSSHVPRFALSNGVLFANCISVTC
QCQTTGRAISQSGEQTLLMIDNTTCPTAVLGNVIISLGKYLGSVNYNSE
GIAIGPPVFTDKVDISSQISSMNQSLQQSKDYIKEAQRLLDTVNPSLKL
MKQIEDKIEEILSKIYHIENEIARIKKLIGEAPGGIEGR

The Nipah virus EphrinB2 receptor is provided as SED ID NO 20:

MAVRRDSVWKYCWGVLMVLCRTAISKSIVLEPIYWNSSNSKFLPGQGLV
LYPQIGDKLDIICPKVDSKTVGQYEYYKVYMVDKDQADRCTIKKENTPL
LNCAKPDQDIKFTIKFQEFSPNLWGLEFQKNKDYYIISTSNGSLEGLDN
QEGGVCQTRAMKILMKVGQDASSAGSTRNKDPTRRPELEAGTNGRSSTT
SPFVKPNPGSSTDGNSAGHSGNNILGSEVALFAGIASGCIIFIVIIITL
VVLLLKYRRRHRKHSPQHTTTLSLSTLATPKRSGNNNGSEPSDIIIPLR
TADSVFCPHYEKVSGDYGHPVYIVQEMPPQSPANIYYKV

The prefusion stabilized F ectodomain of the human metapneumovirus (HMPV) is provided as SEQ ID NO 21:

MSWKVVIIFSLLITPQHGLKESYLEESCSTITEGYLSVLRTGWYTNVFT
LEVGDVENLTCADGPSLIKTELDLTKSALRELRTVSADQLAREEQIENP
RRRRFVLGAIALGVATAAAVTAGVAIAKTIRLESEVTAIKNALKKTNEA
VSTLGNGVRVLATAVRELKDFVSKNLTRAINKNKCDIPDLKMAVSFSQF
NRRFLNVVRQFSDNAGITPAISLDLMTDAELARAVSNMPTSAGQIKLML
ENRAMVRRKGFGILIGVYGSSVIYMVQLPIFGVIDTPCWIVKAAPSCSE
KKGNYACLLREDQGWYCQNAGSTVYYPNEKDCETRGDHVFCDTAAGINV
AEQSKECNINISTTNYPCKVSTGRHPISMVALSPLGALVACYKGVSCSI
GSNRVGIIKQLNKGCSYITNQDADTVTIDNTVYQLSKVEGEQHVIKGRP
VSSSFDPVKFPEDQFNVALDQVFESIENSQALVDQSNRILSSAEKGNTS
GRENLYFQGGGGSGYIPEAPRDGQAYVRKDGEWVLLSTFLGGIEGRHHH
HHH

The prefusion stabilized GPC ectodomain of the Lymphocytic choriomeningitis virus (LCMV) is provided as SEQ ID NO 22:

MGQIVTMFEALPHIIDEVINIVIIVLIIITSIKAVYNFATCGILALVSF
LFLAGRSCGMYGLNGPDIYKGVYQFKSVEFDMSHLNLTMPNACSANNSH
HYISMGSSGLELTFTNDSILNHNFCNLTSAFNKKTFDHTLMSIVSSLHL
SIRGNSNHKAVSCDFNNGITIQYNLSFSDPQSAISQCRTFRGRVLDMFR
TAFGGKYMRSGWGWAGSDGKTTWCSQTSYQYLIIQNRTWENHCRYAGPF
GMSRILFAQEKTKFLTRRLAGTFTWTLSDSSGVENPGGYCLTKWMILAA
ELKCFGNTAVAKCNVNHDEEFCDMLRLIDYNKAALSKFKQDVESALHVF
KTTVNSLISDQLLMRNHLRDLMGVPYCNYSKFWYLEHAKTGETSVPKCW
LVINGSYLNETHFSDQIEQEADNMITEMLRKDYIKRQGSTPLALMD

The LCMV α-dystroglycan (DAG1) receptor is provided as SEQ ID NO 23:

MRMSVGLSLLLPLSGRTFLLLLSVVMAQSHWPSEPSEAVRDWENQLEAS
MHSVLSDLHEAVPTVVGIPDGTAVVGRSFRVTIPTDLIASSGDIIKVSA
AGKEALPSWLHWDSQSHTLEGLPLDTDKGVHYISVSATRLGANGSHIPQ
TSSVFSIEVYPEDHSELQSVRTASPDPGEVVSSACAADEPVTVLTVILD
ADLTKMTPKQRIDLLHRMRSFSEVELHNMKLVPVVNNRLFDMSAFMAGP
GNAKKVVENGALLSWKLGCSLNQNSVPDIHGVEAPAREGAMSAQLGYPV
VGWHIANKKPPLPKRVRRQIHATPTPVTAIGPPTTAIQEPPSRIVPTPT
SPAIAPPTETMAPPVRDPVPGKPTVTIRTRGAIIQTPTLGPIQPTRVSE
AGTTVPGQIRPTMTIPGYVEPTAVATPPTTTTKKPRVSTPKPATPSTDS
TTTTTRRPTKKPRTPRPVPRVTTKVSITRLETASPPTRIRTTTSGVPRG
GEPNQRPELKNHIDRVDAWVGTYFEVKIPSDTFYDHEDTTTDKLKLTLK
LREQQLVGEKSWVQFNSNSQLMYGLPDSSHVGKHEYFMHATDKGGLSAV
DAFEIHVHRRPQGDRAPARFKAKFVGDPALVLNDIHKKIALVKKLAFAF
GDRNCSTITLQNITRGSIVVEWTNNTLPLEPCPKEQIAGLSRRIAEDDG
KPRPAFSNALEPDFKATSITVTGSGSCRHLQFIPVVPPRRVPSEAPPTE
VPDRDPEKSSEDDVYLHTVIPAVVVAAILLIAGIIAMICYRKKRKGKLT
LEDQATFIKKGVPIIFADELDDSKPPPSSSMPLILQEEKAPLPPPEYPN
QSVPETTPLNQDTMGEYTPLRDEDPNAPPYQPPPPFTAPMEGKGSRPKN
MTPYRSPPPYVPP

The prefusion stabilized GPC ectodomain of the Lassa (LASV) Bunyavirus is provided as SEQ ID NO 24:

MRMSVGLSLLLPLSGRTFLLLLSVVMAQSHWPSEPSEAVRDWENQLEAS
MHSVLSDLHEAVPTVVGIPDGTAVVGRSFRVTIPTDLIASSGDIIKVSA
AGKEALPSWLHWDSQSHTLEGLPLDTDKGVHYISVSATRLGANGSHIPQ
TSSVFSIEVYPEDHSELQSVRTASPDPGEVVSSACAADEPVTVLTVILD
ADLTKMTPKQRIDLLHRMRSFSEVELHNMKLVPVVNNRLFDMSAFMAGP
GNAKKVVENGALLSWKLGCSLNQNSVPDIHGVEAPAREGAMSAQLGYPV
VGWHIANKKPPLPKRVRRQIHATPTPVTAIGPPTTAIQEPPSRIVPTPT
SPAIAPPTETMAPPVRDPVPGKPTVTIRTRGAIIQTPTLGPIQPTRVSE
AGTTVPGQIRPTMTIPGYVEPTAVATPPTTTTKKPRVSTPKPATPSTDS
TTTTTRRPTKKPRTPRPVPRVTTKVSITRLETASPPTRIRTTTSGVPRG
GEPNQRPELKNHIDRVDAWVGTYFEVKIPSDTFYDHEDTTTDKLKLTLK
LREQQLVGEKSWVQFNSNSQLMYGLPDSSHVGKHEYFMHATDKGGLSAV
DAFEIHVHRRPQGDRAPARFKAKFVGDPALVLNDIHKKIALVKKLAFAF
GDRNCSTITLQNITRGSIVVEWTNNTLPLEPCPKEQIAGLSRRIAEDDG
KPRPAFSNALEPDFKATSITVTGSGSCRHLQFIPVVPPRRVPSEAPPTE
VPDRDPEKSSEDDVYLHTVIPAVVVAAILLIAGIIAMICYRKKRKGKLT
LEDQATFIKKGVPIIFADELDDSKPPPSSSMPLILQEEKAPLPPPEYPN
QSVPETTPLNQDTMGEYTPLRDEDPNAPPYQPPPPFTAPMEGKGSRPKN
MTPYRSPPPYVPP

The Lassa virus α-dystroglycan (DAG1) receptor has been provided as SEQ ID NO 23.

The stabilized gB protein of the Herpes simplex (HSV) Herepesvirus is provided as SEQ ID NO 25:

MHQGAPSWGRRWFVVWALLGLTLGVLVASAAPSSPGTPGVAAATQAANG
GPATPAPPALGAAPTGDPKPKKNKKPKNPTPPRPAGDNATVAAGHATLR
EHLRDIKAENTDANFYVCPPPTGATVVQFEQPRRCPTRPEGQNYTEGIA
VVFKENIAPYKFKATMYYKDVTVSQVWFGHRYSQFMGIFEDRAPVPFEE
VIDKINAKGVCRSTAKYVRNNLETTAFHRDDHETDMELKPANAATRTSR
GWHTTDLKYNPSRVEAFHRYGTTVNCIVEEVDARSVYPYDEFVLATGDF
VYMSPFYGYREGSHTEHTSYAADRFKQVDGFYARDLTTKARATAPTTRN
LLTTPKFTVAWDWVPKRPSVCTMTKWQEVDEMLRSEYGGSFRFSSDAIS
TTFTTNLTEYPLSRVDLGDCIGKDARDAMDRIFARRYNATHIKVGQPQY
YLANGGFLIAYQPLLSNTLAELYVREHLREQSRKPPNPTPPPPGASANA
SVERIKTTSSIEFARLQFTYNHIQRPVNDMLGRVAIAWCELQNHELTLW
NEARKLNPNAIASVTVGRRVSARMLGDVMAVSTCVPVAADNVIVQNSMR
ISSRPGACYSRPLVSFRYEDQGPLVEGQLGENNELRLTRDAIEPCTVGH
RRYFTFGGGYVYFEEYAYSHQLSRADITTVSTFIDLNITMLEDHEFVPL
EVYTRHEIKDSGLLDYTEVQRRNQLHDLRFADIDTVIHADANAAMFAGL
GAFFEGMGDLGRAVGKVVMGIVGGVVSAVSGVSSFMSNPFGALAVGLLV
LAGLAAAFFAFRYVMRLQSNPMKALYPLTTKELKNPTNPDASGEGEEGG
DFDEAKLAEAREMIRYMALVSAMERTEHKAKKKGTSALLSAKVTDMVMR
KRRNTNYTQVPNKDGDADEDDL

The HBsAg of the Hepatitis B and D (HBV/HDV) Hepadnavirus is provided as SEQ ID NO 26:

MGQNLSTSNPLGFFPDHQLDPAFRANTANPDWDFNPNKDTWPDANKVGA
GAFGLGFTPPHGGLLGWSPQAQGILQTLPANPPPASTNRQSGRQPTPLS
PPLRNTHPQAMQWNSTTFHQTLQDPRVRGLYFPAGGSSSGTVNPVLTTA
SPLSSIFSRIGDPALNMENITSGFLGPLLVLQAGFFLLTRILTIPQSLD
SWWTSLNFLGGTTVCLGQNSQSPTSNHSPTSCPPTCPGYRWMCLRRFII
FLFILLLCLIFLLVLLDYQGMLPVCPLIPGSSTTSTGPCRTCMTTAQGT
SMYPSCCCTKPSDGNCTCIPIPSSWAFGKFLWEWASARFSWLSLLVPFV
QWFVGLSPTVWLSVIWMMWYWGPSLYSILSPFLPLLPIFFCLWVYI

The HBV/HDV sodium/taurocholate cotransporting polypeptide (NTCP) receptor is provided as SEQ ID NO 27:

MEAHNASAPFNFTLPPNFGKRPTDLALSVILVFMLFFIMLSLGCTMEFS
KIKAHLWKPKGLAIALVAQYGIMPLTAFVLGKVFRLKNIEALAILVCGC
SPGGNLSNVFSLAMKGDMNLSIVMTTCSTFCALGMMPLLLYIYSRGIYD
GDLKDKVPYKGIVISLVLVLIPCTIGIVLKSKRPQYMRYVIKGGMIIIL
LCSVAVTVLSAINVGKSIMFAMTPLLIATSSLMPFIGFLLGYVLSALFC
LNGRCRRTVSMETGCQNVQLCSTILNVAFPPEVIGPLFFFPLLYMIFQL
GEGLLLIAIFWCYEKFKTPKDKTKMIYTAATTEETIPGALGNGTYKGED
CSPCTA

The E2 ectodomain of the Hepatitis C (HCV) Flavivirus is provided as SEQ ID NO 28:

NQLINTNGSWHLNRTALNCNDSLNTGWLAGLFYHHKFDSSGCPERMASC
RPLTDFDQGWGPISHANGSGPDGSSGCWHYPPRPCGIVPAKTVCGPVYC
FTPSPVVVGTTDRAGAPAYNWGENDTDVFVLNNTRPPLGNWFGCTWMNS
TGFTKACGAPPCAIGGVGDKTLYCPTDCFRKHPEATYSRCGSGPWITPR
CLVDYPYRLWHYPCTINYTVFKIRMYVGGVEHRLEAACN

The HCV CD81 antigen receptor is provided as SEQ ID NO 29:

MGVEGCTKCIKYLLFVFNFVFWLAGGVILGVALWLRHDPQTTNLLYLEL
GDKPAPNTFYVGIYILIAVGAVMMFVGFLGCYGAIQESQCLLGTFFTCL
VILFACEVAAGIWGFVNKDQIAKDVKQFYDQALQQAVVDDDANNAKAVV
KTFHETLDCCGSSTLTALTTSVLKNNLCPSGSNIISNLFKEDCHQKIDD
LFSGKLYLIGIAAIVVAVIMIFEMILSMVLCCGIRNSSVY

In one embodiment of the method of the invention, the prefusion viral protein is selected from the group comprising SEQ ID NO: 1, SEQ ID NO: 3, SEQ ID NO: 5, SEQ ID NO: 7, SEQ ID NO: 9, SEQ ID NO: 11, SEQ ID NO: 12, SEQ ID NO: 14, SEQ ID NO: 15, SEQ ID NO: 17, SEQ ID NO: 19, SEQ ID NO: 21, SEQ ID NO: 22, SEQ ID NO: 24, SEQ ID NO: 25, SEQ ID NO: 26, SEQ ID NO: 28, SEQ ID NO: 30, SEQ ID NO: 31, SEQ ID NO: 32, SEQ ID NO: 33, SEQ ID NO: 34, SEQ ID NO: 35, and SEQ ID NO: 36, and the cell receptor protein is selected from the group comprising SEQ ID NO: 2, SEQ ID NO: 4, SEQ ID NO: 6, SEQ ID NO: 8, SEQ ID NO: 10, SEQ ID NO: 13, SEQ ID NO: 16, SEQ ID NO: 18, SEQ ID NO: 20, SEQ ID NO: 23, SEQ ID NO: 27, and SEQ ID NO: 29. The cell receptor is expressed by the cell susceptible to virus infection.

In another embodiment of the method of the invention, the prefusion viral protein consists of or comprises the amino acid sequence or at least 90%, 95%, 96%, 97%, 98% or 99% sequence identity thereto, selected from the group comprising SEQ ID NO: 1, SEQ ID NO: 3, SEQ ID NO: 5, SEQ ID NO: 7, SEQ ID NO: 9, SEQ ID NO: 11, SEQ ID NO: 12, SEQ ID NO: 14, SEQ ID NO: 15, SEQ ID NO: 17, SEQ ID NO: 19, SEQ ID NO: 21, SEQ ID NO: 22, SEQ ID NO: 24, SEQ ID NO: 25, SEQ ID NO: 26, SEQ ID NO: 28, SEQ ID NO: 30, SEQ ID NO: 31, SEQ ID NO: 32, SEQ ID NO: 33, SEQ ID NO: 34, SEQ ID NO: 35, SEQ ID NO: 36, and the cell receptor protein consists of or comprises the amino acid sequence or at least 90%, 95%, 96%, 97%, 98% or 99% sequence identity thereto, selected from the group comprising SEQ ID NO: 2, SEQ ID NO: 4, SEQ ID NO: 6, SEQ ID NO: 8, SEQ ID NO: 10, SEQ ID NO: 13, SEQ ID NO: 16, SEQ ID NO: 18, SEQ ID NO: 20, SEQ ID NO: 23, SEQ ID NO: 27, SEQ ID NO: 29. The cell receptor is expressed by the cell susceptible to virus infection.

In another embodiment of the method of the invention, the prefusion viral protein consists of or comprises the amino acid sequence selected from the group comprising SEQ ID NO: 1, 30, 31, 32, 33, 34, 35 and 36, and the cell receptor protein consists of or comprises SEQ ID NO: 2.

In a further embodiment of the method of the invention, the prefusion viral protein consists of or comprises the amino acid sequence or at least 90%, 95%, 96%, 97%, 98% or 99% sequence identity thereto, selected from the group comprising SEQ ID NO: 1, SEQ ID NO: 30, SEQ ID NO: 31, SEQ ID NO: 32, SEQ ID NO: 33, SEQ ID NO: 34, SEQ ID NO: 35, and SEQ ID NO: 36 and the cell receptor protein consists of or comprises the amino acid sequence SEQ ID NO: 2 or at least 90%, 95%, 96%, 97%, 98% or 99% sequence identity thereto.

In the method of the invention, the pairs “prefusion viral protein” and “cell receptor protein” used in the method are for example the following: SEQ ID NO: 1, 30, 31, 32, 33, 34, 35 and/or 36 and SEQ ID NO: 2; SEQ ID NO: 3 and SEQ ID NO: 4; SEQ ID NO: 5 and SEQ ID NO: 6; SEQ ID NO: 7 and SEQ ID NO: 8; SEQ ID NO: 9 and SEQ ID NO: 10; SEQ ID NO: 12 and SEQ ID NO: 13; SEQ ID NO: 14-15 and SEQ ID NO: 16; SEQ ID NO: 17 and SEQ ID NO: 18; SEQ ID NO: 19 and SEQ ID NO: 20; SEQ ID NO: 22 and SEQ ID NO: 23; SEQ ID NO: 24 and SEQ ID NO: 23; SEQ ID NO: 26 and SEQ ID NO: 27; SEQ ID NO: 28 and SEQ ID NO: 29. The cell receptor is expressed by the cell susceptible to virus infection.

In some embodiments of the method of the invention, the prefusion viral protein and the cell receptor protein that binds to the prefusion viral protein (provide pairs “prefusion viral protein” and “cell receptor protein”) are selected from the group comprising
a) the prefusion viral protein consisting of or comprising the amino acid sequence or at least 90%, 95%, 96%, 97%, 98% or 99% sequence identity thereto, selected from the group comprising SEQ ID NOs:1, 30, 31, 32, 33, 34, 35 and 36, and the cell receptor protein consisting of or comprising SEQ ID NO:2 or at least 90%, 95%, 96%, 97%, 98% or 99% sequence identity thereto,
b) the prefusion viral protein consisting of or comprising SEQ ID NO:3 or at least 90%, 95%, 96%, 97%, 98% or 99% sequence identity thereto, and the cell receptor protein consisting of or comprising SEQ ID NO:4 or at least 90%, 95%, 96%, 97%, 98% or 99% sequence identity thereto,
c) the prefusion viral protein consisting of or comprising SEQ ID NO:5 or at least 90%, 95%, 96%, 97%, 98% or 99% sequence identity thereto, and the cell receptor protein consisting of or comprising SEQ ID NO:6 or at least 90%, 95%, 96%, 97%, 98% or 99% sequence identity thereto,
d) the prefusion viral protein consisting of or comprising SEQ ID NO:7 or at least 90%, 95%, 96%, 97%, 98% or 99% sequence identity thereto, and the cell receptor protein consisting of or comprising SEQ ID NO:8 or at least 90%, 95%, 96%, 97%, 98% or 99% sequence identity thereto,
e) the prefusion viral protein consisting of or comprising SEQ ID NO:9 or at least 90%, 95%, 96%, 97%, 98% or 99% sequence identity thereto, and the cell receptor protein consisting of or comprising SEQ ID NO:10 or at least 90%, 95%, 96%, 97%, 98% or 99% sequence identity thereto,
f) the prefusion viral protein consisting of or comprising SEQ ID NO:12 or at least 90%, 95%, 96%, 97%, 98% or 99% sequence identity thereto, and the cell receptor protein consisting of or comprising SEQ ID NO:13 or at least 90%, 95%, 96%, 97%, 98% or 99% sequence identity thereto,
g) the prefusion viral protein consisting of or comprising SEQ ID NO:14 or SEQ ID NO:15 or at least 90%, 95%, 96%, 97%, 98% or 99% sequence identity thereto, and the cell receptor protein consisting of or comprising SEQ ID NO:16 or at least 90%, 95%, 96%, 97%, 98% or 99% sequence identity thereto,
h) the prefusion viral protein consisting of or comprising SEQ ID NO:17 or at least 90%, 95%, 96%, 97%, 98% or 99% sequence identity thereto, and the cell receptor protein consisting of or comprising SEQ ID NO:18 or at least 90%, 95%, 96%, 97%, 98% or 99% sequence identity thereto,
i) the prefusion viral protein consisting of or comprising SEQ ID NO:19 or at least 90%, 95%, 96%, 97%, 98% or 99% sequence identity thereto, and the cell receptor protein consisting of or comprising SEQ ID NO:20 or at least 90%, 95%, 96%, 97%, 98% or 99% sequence identity thereto,
j) the prefusion viral protein consisting of or comprising SEQ ID NO:22 or at least 90%, 95%, 96%, 97%, 98% or 99% sequence identity thereto, and the cell receptor protein consisting of or comprising SEQ ID NO:23 or at least 90%, 95%, 96%, 97%, 98% or 99% sequence identity thereto,
k) the prefusion viral protein consisting of or comprising SEQ ID NO:24 or at least 90%, 95%, 96%, 97%, 98% or 99% sequence identity thereto, and the cell receptor protein consisting of or comprising SEQ ID NO:23 or at least 90%, 95%, 96%, 97%, 98% or 99% sequence identity thereto,
l) the prefusion viral protein consisting of or comprising SEQ ID NO:26 or at least 90%, 95%, 96%, 97%, 98% or 99% sequence identity thereto, and the cell receptor protein consisting of or comprising SEQ ID NO:27 or at least 90%, 95%, 96%, 97%, 98% or 99% sequence identity thereto,
m) the prefusion viral protein consisting of or comprising SEQ ID NO:28 or at least 90%, 95%, 96%, 97%, 98% or 99% sequence identity thereto, and the cell receptor protein consisting of or comprising SEQ ID NO:29 or at least 90%, 95%, 96%, 97%, 98% or 99% sequence identity thereto.

In an embodiment of the method for quantifying virus neutralizing antibodies of the invention, the at least one prefusion viral proteins is one or more viral prefusion proteins, two or more viral prefusion proteins, such as 2, 3, 4, 5, 6, 7, 8, 9, 10, 11, 12, 13, 14, 15, 16, 17, 18, 19 or 20 viral prefusion proteins.

In an embodiment of the method for quantifying virus neutralizing antibodies of the invention, the virus is SARS-CoV-2 virus and the at least one prefusion viral protein(s) is/are the at least one prefusion SARS-CoV-2 protein(s) (such as prefusion trimeric SARS-CoV-2 spike protein(s)), the cell receptor protein is ACE-2-Fc fusion protein, and the one or more competing antibodies bind to the at least one prefusion SARS-CoV-2 protein(s). According to a preferred embodiment, the at least one prefusion SARS-CoV-2 protein(s) is/are one or more (or two or more) prefusion SARS-CoV-2 proteins consisting of or comprising the amino acid sequence or at least 90%, 95%, 96%, 97%, 98% or 99% sequence identity thereto, selected from the group consisting of SEQ ID NO: 1, 30, 31, 32, 33, 34, 35 and 36, and the ACE-2-Fc fusion protein consists of or comprises SEQ ID NO: 2 or at least 90%, 95%, 96%, 97%, 98% or 99% sequence identity thereto. In another preferred embodiment, the at least one prefusion SARS-CoV-2 proteins are 8 prefusion SARS-CoV-2 proteins consisting of or comprising the amino acid sequences or at least 90%, 95%, 96%, 97%, 98% or 99% sequence identity thereto, selected from the group consisting of SEQ ID NO: 1, 30, 31, 32, 33, 34, 35 and 36, and the ACE-2-Fc fusion protein consists of or comprises SEQ ID NO: 2 or at least 90%, 95%, 96%, 97%, 98% or 99% sequence identity thereto.

In one implementation of the method for quantifying SARS-CoV-2 neutralizing antibodies in a biological sample of a subject, the spike trimer of SARS-CoV-2 is coupled to Luminex beads, which are exposed to soluble ACE2 cell receptor in the presence of limited dilutions of tested serum. Amounts of captured ACE2 are then measured by direct or indirect methods (e.g. with a fluorescence-coupled ACE2-specific antibody).

An embodiment of the invention provides the method for quantifying SARS-CoV-2 virus neutralizing antibodies in a biological sample of a subject, comprising
a) providing at least one prefusion SARS-CoV-2 protein, that is/are stabilized in a prefusion conformation and that is reactive with the SARS-CoV-2 virus neutralizing antibodies, and coupling said at least one prefusion SARS-CoV-2 protein(s) to a at least one solid support,
b) obtaining a biological sample from a subject,
c) contacting (mixing) the at least one prefusion SARS-CoV-2 protein(s) coupled to the at least one solid support with the biological sample to provide a mixture,
d) contacting (adding to) the mixture with ACE-2-Fc fusion protein, that binds to the at least one prefusion SARS-CoV-2 protein or a competing antibody that binds to the at least one prefusion SARS-CoV-2 protein,
e) detecting and quantifying SARS-CoV-2 virus neutralizing antibodies in the biological sample.

In a further embodiment of the method of the invention, the prefusion SARS-CoV-2 protein consists of, or comprises, a multiplex of different variants of S proteins consisting of or comprising the amino acid sequences or at least 90%, 95%, 96%, 97%, 98% or 99% sequence identity thereto, selected from the group comprising SEQ ID NO: 1, 30, 31, 32, 33, 34, 35 and/or 36 in prefusion configuration. In a specific embodiment, the multiplex of S protein is a trimer (S³) in prefusion configuration. The method of the invention is designed and adapted to include any new variant of prefusion SARS-CoV-2 protein (prefusion SARS-CoV-2 spike protein) that can emerge.

According to a preferred embodiment of the method of the invention, the at least one prefusion SARS-CoV-2 protein(s) is/are one or more (or two or more) prefusion SARS-CoV-2 proteins consisting of or comprising the amino acid sequence or at least 90%, 95%, 96%, 97%, 98% or 99% sequence identity thereto, selected from the group consisting of SEQ ID NO: 1, 30, 31, 32, 33, 34, 35 and 36, and the ACE-2-Fc fusion protein consists of or comprises SEQ ID NO: 2 or at least 90%, 95%, 96%, 97%, 98% or 99% sequence identity thereto. In another preferred embodiment, the at least one prefusion SARS-CoV-2 proteins are 8 prefusion SARS-CoV-2 proteins consisting of or comprising the amino acid sequences or at least 90%, 95%, 96%, 97%, 98% or 99% sequence identity thereto, selected from the group consisting of SEQ ID NO: 1, 31, 32, 33, 34, 35 and 36, and the ACE-2-Fc fusion protein consists of or comprises SEQ ID NO: 2 or at least 90%, 95%, 96%, 97%, 98% or 99% sequence identity thereto.

The method of the invention can be used for evaluating immune protection after natural virus infection, such as SARS-CoV-2 infection; evaluating immune protection after administration of a vaccine, such as SARS-CoV-2 vaccine; testing therapeutic potential of post-recovery plasma, serum or purified antibodies; testing therapeutic potential of monoclonal antibodies; testing therapeutic potential of other molecules interfering with envelope-receptor or virus-target cell interactions.

In some embodiments of the invention, the method for quantifying virus neutralizing antibodies in a biological sample of a subject can be used to evaluate immune protection after natural virus infection, such as SARS-CoV-2 virus, and decide whether administration of a vaccine (such as a vaccine against SARS-CoV-2 virus) is needed. The administration of a vaccine may be needed if the subject does not have (or has very low concentration) of neutralizing antibodies compared to other subjects that have been infected by the virus, such as SARS-CoV-2 virus. In some other embodiments, the method for quantifying virus neutralizing antibodies in a biological sample of a subject can be used to evaluate immune protection after administration of a vaccine (such as a vaccine against SARS-CoV-2 virus) and decide whether the subject responds to the vaccination or whether the vaccination should be repeated. For example, some subjects may require two, three, four or more doses of the vaccine in order to acquire immune response or some subjects may not respond to vaccination such as primary or acquired immunodeficiency patients and/or cancer, transplant and patients with systemic inflammatory diseases receiving immunosuppressive treatments.

According to an embodiment, the method of the present invention relates to multiplex method to simultaneously quantify neutralizing antibodies against viruses or variants of viruses (i.e. measure concentrations or titer of virus neutralizing antibodies) present in a biological sample. A method of using the multiplex method to quantify virus neutralizing antibodies following viral infection and/or vaccination against viruses or variants of viruses, such as SARS-CoV-2 virus, is also contemplated. Thus according to an embodiment, the method of the invention is a multiplex quantifying method of virus neutralizing antibodies, comprising quantifying neutralizing antibodies targeting two or more variants simultaneously.

The multiplex method (or assay) of the present invention is a type of immunoassay that uses solid supports, such as beads or microspheres (for example Luminex beads or microspheres) to simultaneously measure neutralizing antibodies targeting multiple variants in a single experiment. In the multiplex method, a solid support, such as beads or microspheres (for example Luminex beads or microspheres) of a designated color is coated with a prefusion viral protein or variant thereof of defined susceptibility to neutralizing antibodies. The results can be read by flow cytometry because each set of beads coupled with a specific protein or variant thereof is distinguishable from the other by its fluorescent signature. The number of viral proteins or variants thereof tested simultaneously for their resistance to serum or antibodies neutralization is determined by the number of different beads colors used.

In an embodiment of the method of the invention, the at least one prefusion viral protein(s) consists of, or comprises, a multiplex of different variants of the same prefusion viral proteins or of different prefusion viral proteins, such as from different viruses.

In a further embodiment of the method of the invention, the at least one prefusion viral protein(s) consists of, or comprises, a multiplex of same or different S proteins in prefusion configuration. In a specific embodiment, the multiplex of S protein is a trimer (S³) of the ectodomain in a stabilized prefusion configuration.

According to the present invention, the combination of two or more different prefusion viral proteins for the simultaneous detection and quantification of neutralizing antibodies is termed “multiplex” or “multiplexing”.

The method for quantifying virus neutralizing antibodies of the present invention is based on the competitive inhibition of a cell receptor (such as ACE2) binding to prefusion viral protein bound to a solid support (such as S protein trimers-bearing beads). Indeed, it relies on the fact that most neutralizing antibodies interfere with the binding of the prefusion viral proteins, such as viral S protein, with its cell receptor protein, such as ACE2 receptor. Although neutralizing antibodies in case of SARS-CoV-2 virus have been identified that recognize S protein outside of the RBD and do not directly impact on ACE2 binding, these are rare and likely contribute minimally to antiviral immunity, as confirmed by the very high degree of correlation between our surrogate assay and its live virus cell-based reference counterpart, irrespective of the levels of neutralizing activity. The method of the invention is high-throughput, quantitative, yields results tightly correlating those obtained with a classical wild-type virus cell-based neutralization assay, and allows the simultaneous evaluation of multiple variants of prefusion viral proteins (such as multiple S protein variants). Illustrating its value, it reveals that some previously infected individuals display variant-specific serum neutralizing antibody titers, suggesting that they remain at risk of infection with at least some VOCs.

The method for quantifying virus neutralizing antibodies of the present invention allows for the quantitative and high-throughput evaluation of the neutralizing activity of biological samples such as serum against multiple prefusion viral proteins, such as multiple SARS-CoV-2 variants, in a single procedure taking less than three hours in a standard diagnostic laboratory. The method for quantifying virus neutralizing antibodies of the present invention provides quantitative measures of serum neutralizing antibody levels and is further characterized by a very high degree of sensitivity and specificity (>96% and 100%, respectively, using the herein described protocol).

The advantage of the method of the invention over the prior art is use of a prefusion (ectodomain) viral protein closely mimicking the protein found on the surface of virions (for example stabilized trimers, in prefusion configuration), and by its ability to yield quantitative results regarding neutralizing antibody titers.

A significant advantage of the S³-ACE2 neutralization assay according to the present invention is its ability to evaluate multiple S variants in parallel using as little at 15 μl of serum, allowing the identification of an individual's susceptibility to circulating and emerging SARS-CoV-2 viruses, whether after infection or vaccination. This was demonstrated with serum samples from 59 COVID-19 patients infected prior to the widespread emergence of VOCs. Importantly, only 43% of non-ICU hospitalized patients had neutralizing antibody levels greater than 50 serum dilution IC₅₀ against the S protein with the K417N/E484K/N501Y mutation found in the B.1.351 variant, suggesting that many are not protected against this strain. The P.1 variant originally identified in Brazil has a similar mutation profile in the RBD and some of these non-ICU patients would be anticipated to be equally susceptible to infection by this SARS-CoV-2 variant as well. Individuals whose infection had required an ICU stay generally displayed higher levels of neutralizing antibodies against all tested S protein variants, although they too were less effective against the B1.351/P1 triple mutant.

The multiplexing of the S³-ACE2 neutralization assay according to the present invention can be increased to >40 different S alleles to tackle new VOCs. It advantageously compares with viral pseudotypes-based systems, where each S protein mutant requires production of a new batch of virions that have to be tested in separate assays, with their infectivity potentially affected by the mutations and neutralization titers influenced by the levels of ACE2 on the surface of target cells. As levels of anti-SARS-CoV-2 immunity increase in the world population due to the combined influence of ongoing infections and more widespread vaccination, selective pressures will increasingly be exerted on the virus favoring the emergence of escape mutants. The detection of these escapees should be as fast as possible for the swift adaptation of prophylactic measures including vaccines. While the infection of previously infected or vaccinated individuals will remain the strongest evidence of gaps in the collective immunity, a surveillance system based on the routine sequencing of viral isolates and the immediate testing of the susceptibility of their S protein to neutralization would constitute a far more dynamic and anticipatory approach. The S³-ACE2 neutralization assay according to the present invention, because of its ease of use, would facilitate such surveillance strategy. Furthermore, it could also be used for the high-throughput screening of candidate monoclonal antibodies and other prophylactic or therapeutic approaches aimed at blocking the interaction between SARS-CoV-2 and its cellular receptor, and it could be adapted to other viruses for which the molecular mediators of viral entry are properly characterized.

Therefore, the hereby described method for quantifying virus neutralizing antibodies stands to have an important impact in both clinical and public health settings. In this regard, immunity passports are at the forefront of current public and political discussions as possible gateways to a return to more normal social and international exchanges, as the world emerges from the COVID-19 pandemics. They are generally thought of essentially as vaccination certificates, a concept that suffers from major shortcomings. First, such certificates would unduly exclude people that have not yet been vaccinated but endowed with strong antiviral immunity triggered by natural infection. Second, they would not be delivered to individuals that do not respond to vaccination such as primary or acquired immunodeficiency patients and/or cancer, transplant and patients with systemic inflammatory diseases receiving immunosuppressive treatments. These individuals could however be protected by passive immunization through the administration of human monoclonal antibodies, the activity of which could be quantified in their serum. Third, having been vaccinated is not a guarantee of induction of optimal immunity and protection, as found every year with the flu vaccine, and the duration of vaccine-induced SARS-CoV-2 immunity is as yet unknown.

The method of the invention can be also used for screening a neutralizing antibody to virus, such as SARS-CoV-2 virus, to identify and/or quantify neutralizing antibodies (measure neutralizing antibody titer).

Another aspect of the present invention provides a kit comprising
at least one prefusion viral protein(s), that is stabilized in a prefusion conformation and that is reactive with neutralizing antibodies,
one or more cell receptor proteins that bind to the at least one prefusion viral protein or one or more competing antibody that binds to the at least one prefusion viral protein(s), and
instructions for use.

In one embodiment of the kit of the invention, the prefusion viral protein is selected from the group comprising SEQ ID NO: 1, SEQ ID NO: 3, SEQ ID NO: 5, SEQ ID NO: 7, SEQ ID NO: 9, SEQ ID NO: 11, SEQ ID NO: 12, SEQ ID NO: 14, SEQ ID NO: 15, SEQ ID NO: 17, SEQ ID NO: 19, SEQ ID NO: 21, SEQ ID NO: 22, SEQ ID NO: 24, SEQ ID NO: 25, SEQ ID NO: 26, SEQ ID NO: 28, SEQ ID NO: 30, SEQ ID NO: 31, SEQ ID NO: 32, SEQ ID NO: 33, SEQ ID NO: 34, SEQ ID NO: 35, SEQ ID NO: 36, and the cell receptor protein is selected from the group comprising SEQ ID NO: 2, SEQ ID NO: 4, SEQ ID NO: 6, SEQ ID NO: 8, SEQ ID NO: 10, SEQ ID NO: 13, SEQ ID NO: 16, SEQ ID NO: 18, SEQ ID NO: 20, SEQ ID NO: 23, SEQ ID NO: 27, SEQ ID NO: 29.

In another embodiment of the kit of the invention, the prefusion viral protein consists of or comprises amino acid sequence or at least 90%, 95%, 96%, 97%, 98% or 99% sequence identity thereto, selected from the group comprising SEQ ID NO: 1, SEQ ID NO: 3, SEQ ID NO: 5, SEQ ID NO: 7, SEQ ID NO: 9, SEQ ID NO: 11, SEQ ID NO: 12, SEQ ID NO: 14, SEQ ID NO: 15, SEQ ID NO: 17, SEQ ID NO: 19, SEQ ID NO: 21, SEQ ID NO: 22, SEQ ID NO: 24, SEQ ID NO: 25, SEQ ID NO: 26, SEQ ID NO: 28, SEQ ID NO: 30, SEQ ID NO: 31, SEQ ID NO: 32, SEQ ID NO: 33, SEQ ID NO: 34, SEQ ID NO: 35, SEQ ID NO: 36, and the cell receptor proteins consists of or comprises amino acid sequence or at least 90%, 95%, 96%, 97%, 98% or 99% sequence identity thereto, selected from the group comprising SEQ ID NO: 2, SEQ ID NO: 4, SEQ ID NO: 6, SEQ ID NO: 8, SEQ ID NO: 10, SEQ ID NO: 13, SEQ ID NO: 16, SEQ ID NO: 18, SEQ ID NO: 20, SEQ ID NO: 23, SEQ ID NO: 27, SEQ ID NO: 29.

In another embodiment of the kit of the invention, the prefusion viral protein consists of or comprises the amino acid sequence selected from the group comprising SEQ ID NO: 1, 30, 31, 32, 33, 34, 35 and 36, and the cell receptor protein consists of or comprises SEQ ID NO: 2.

In a further embodiment of the kit of the invention, the prefusion viral protein consists of or comprises amino acid sequences or at least 90%, 95%, 96%, 97%, 98% or 99% sequence identity thereto, selected from the group comprising SEQ ID NO: 1, SEQ ID NO: 30, SEQ ID NO: 31, SEQ ID NO: 32, SEQ ID NO: 33, SEQ ID NO: 34, SEQ ID NO: 35, and SEQ ID NO: 36, and the cell receptor protein consists of or comprises the amino acid sequence SEQ ID NO: 2 or at least 90%, 95%, 96%, 97%, 98% or 99% sequence identity thereto.

In some embodiments, the kit of the invention is used for quantifying neutralizing antibodies against viruses, such as SARS-CoV-2, typically in the context of a recent, current or prior virus infection or exposure to a virus antigen or polypeptide in a subject susceptible thereto.

The kit of the invention can further comprise other appropriate reagents, preferably a means for detecting/quantifying when neutralizing antibody is bound. For example, the prefusion viral protein or the cell receptor protein may be labelled with a detection means that allows for the detection and quantification of the prefusion viral protein when it is bound to a neutralizing antibody.

Another aspect of the present invention provides a method for measuring the ability of an agent to block entry of a virus into a cell susceptible to virus infection and thereby to inhibit infection of the cell with the virus, wherein the virus is enveloped virus and wherein the method comprises
a) providing at least one prefusion viral protein, that is stabilized in a prefusion conformation and/or that is reactive with the virus neutralizing antibodies, and coupling said prefusion viral protein to at least one solid support,
b) contacting the at least one prefusion viral protein coupled to the at least one solid support with the agent to provide a mixture,
c) contacting (adding to) the mixture with at least one cell receptor protein that binds to the at least one prefusion viral protein,
d) measuring the binding of the cell receptor to the prefusion viral protein to provide an indirect measure of the ability of the agent to inhibit entry of the virus into the cell.

In one embodiment of the method of the invention, the virus is selected from the group comprising Flavivirus, Alphavirus, Coronavirus, Hepatitis D, Orthomyxovirus, Paramyxovirus, Rhabdovirus, Bunyavirus, Filovirus, Retroviruses, Herpesviruses, Poxviruses, Hepadnaviruses, Asfarviridae.

In another embodiment of the method of the invention, the virus is SARS-CoV-2 virus.

In an embodiment of the method of the invention, the agent can be an inhibitor, a blocker, or an antagonist protein, e.g., an antibody or portion of the antibody, a peptide, or a non-protein organic small molecule that inhibits or blocks the interaction or association of the prefusion viral protein with the cell receptor expressed by the cell susceptible to virus infection. An agent that antagonizes the infection or entry of a virus into a cell also may be considered to inhibit, block, or disrupt the infection or entry of a virus into the cell.

In another embodiment of the method of the invention, the cell susceptible to virus infection is human or animal cell.

In one embodiment of the method of the invention, the prefusion viral protein is selected from the group comprising SEQ ID NO: 1, SEQ ID NO: 3, SEQ ID NO: 5, SEQ ID NO: 7, SEQ ID NO: 9, SEQ ID NO: 11, SEQ ID NO: 12, SEQ ID NO: 14, SEQ ID NO: 15, SEQ ID NO: 17, SEQ ID NO: 19, SEQ ID NO: 21, SEQ ID NO: 22, SEQ ID NO: 24, SEQ ID NO: 25, SEQ ID NO: 26, SEQ ID NO: 28, SEQ ID NO: 30, SEQ ID NO: 31, SEQ ID NO: 32, SEQ ID NO: 33, SEQ ID NO: 34, SEQ ID NO: 35, SEQ ID NO: 36 and the cell receptor protein is selected from the group comprising SEQ ID NO: 2, SEQ ID NO: 4, SEQ ID NO: 6, SEQ ID NO: 8, SEQ ID NO: 10, SEQ ID NO: 13, SEQ ID NO: 16, SEQ ID NO: 18, SEQ ID NO: 20, SEQ ID NO: 23, SEQ ID NO: 27, SEQ ID NO: 29. The cell receptor is expressed by the cell susceptible to virus infection.

In another embodiment of the method of the invention, the prefusion viral protein consists of or comprises the amino acid sequence or at least 90%, 95%, 96%, 97%, 98% or 99% sequence identity thereto, selected from the group comprising SEQ ID NO: 1, SEQ ID NO: 3, SEQ ID NO: 5, SEQ ID NO: 7, SEQ ID NO: 9, SEQ ID NO: 11, SEQ ID NO: 12, SEQ ID NO: 14, SEQ ID NO: 15, SEQ ID NO: 17, SEQ ID NO: 19, SEQ ID NO: 21, SEQ ID NO: 22, SEQ ID NO: 24, SEQ ID NO: 25, SEQ ID NO: 26, SEQ ID NO: 28, SEQ ID NO: 30, SEQ ID NO: 31, SEQ ID NO: 32, SEQ ID NO: 33, SEQ ID NO: 34, SEQ ID NO: 35, SEQ ID NO: 36, and the cell receptor protein consists of or comprises the amino acid sequence or at least 90%, 95%, 96%, 97%, 98% or 99% sequence identity thereto, selected from the group comprising SEQ ID NO: 2, SEQ ID NO: 4, SEQ ID NO: 6, SEQ ID NO: 8, SEQ ID NO: 10, SEQ ID NO: 13, SEQ ID NO: 16, SEQ ID NO: 18, SEQ ID NO: 20, SEQ ID NO: 23, SEQ ID NO: 27, SEQ ID NO: 29. The cell receptor is expressed by the cell susceptible to virus infection.

In another embodiment of the method of the invention, the prefusion viral protein consists of or comprises the amino acid sequence selected from the group comprising SEQ ID NO: 1, 30, 31, 32, 33, 34, 35 and 36, and the cell receptor protein consists of or comprises SEQ ID NO: 2.

In a further embodiment of the method of the invention, the prefusion viral protein consists of or comprises the amino acid sequence or at least 90%, 95%, 96%, 97%, 98% or 99% sequence identity thereto, selected from the group comprising SEQ ID NO: 1, SEQ ID NO: 30, SEQ ID NO: 31, SEQ ID NO: 32, SEQ ID NO: 33, SEQ ID NO: 34, SEQ ID NO: 35, and SEQ ID NO: 36, and the cell receptor protein consists of or comprises the amino acid sequence SEQ ID NO: 2 or at least 90%, 95%, 96%, 97%, 98% or 99% sequence identity thereto.

In some embodiments of the method of the invention, the at least one prefusion viral protein and the at least one cell receptor protein that binds to the at least one prefusion viral protein (provide pairs “prefusion viral protein” and “cell receptor protein”) are selected from the group comprising
a) the prefusion viral protein consisting of or comprising the amino acid sequence or at least 90%, 95%, 96%, 97%, 98% or 99% sequence identity thereto, selected from the group comprising SEQ ID NOs:1, 30, 31, 32, 33, 34, 35 and 36, and the cell receptor protein consisting of or comprising SEQ ID NO:2 or at least 90%, 95%, 96%, 97%, 98% or 99% sequence identity thereto,
b) the prefusion viral protein consisting of or comprising SEQ ID NO:3 or at least 90%, 95%, 96%, 97%, 98% or 99% sequence identity thereto, and the cell receptor protein consisting of or comprising SEQ ID NO:4 or at least 90%, 95%, 96%, 97%, 98% or 99% sequence identity thereto,
c) the prefusion viral protein consisting of or comprising SEQ ID NO:5 or at least 90%, 95%, 96%, 97%, 98% or 99% sequence identity thereto, and the cell receptor protein consisting of or comprising SEQ ID NO:6 or at least 90%, 95%, 96%, 97%, 98% or 99% sequence identity thereto,
d) the prefusion viral protein consisting of or comprising SEQ ID NO:7 or at least 90%, 95%, 96%, 97%, 98% or 99% sequence identity thereto, and the cell receptor protein consisting of or comprising SEQ ID NO:8 or at least 90%, 95%, 96%, 97%, 98% or 99% sequence identity thereto,
e) the prefusion viral protein consisting of or comprising SEQ ID NO:9 or at least 90%, 95%, 96%, 97%, 98% or 99% sequence identity thereto, and the cell receptor protein consisting of or comprising SEQ ID NO:10 or at least 90%, 95%, 96%, 97%, 98% or 99% sequence identity thereto,
f) the prefusion viral protein consisting of or comprising SEQ ID NO:12 or at least 90%, 95%, 96%, 97%, 98% or 99% sequence identity thereto, and the cell receptor protein consisting of or comprising SEQ ID NO:13 or at least 90%, 95%, 96%, 97%, 98% or 99% sequence identity thereto,
g) the prefusion viral protein consisting of or comprising SEQ ID NO:14 or SEQ ID NO:15 or at least 90%, 95%, 96%, 97%, 98% or 99% sequence identity thereto, and the cell receptor protein consisting of or comprising SEQ ID NO:16 or at least 90%, 95%, 96%, 97%, 98% or 99% sequence identity thereto,
h) the prefusion viral protein consisting of or comprising SEQ ID NO:17 or at least 90%, 95%, 96%, 97%, 98% or 99% sequence identity thereto, and the cell receptor protein consisting of or comprising SEQ ID NO:18 or at least 90%, 95%, 96%, 97%, 98% or 99% sequence identity thereto,
i) the prefusion viral protein consisting of or comprising SEQ ID NO:19 or at least 90%, 95%, 96%, 97%, 98% or 99% sequence identity thereto, and the cell receptor protein consisting of or comprising SEQ ID NO:20 or at least 90%, 95%, 96%, 97%, 98% or 99% sequence identity thereto,
j) the prefusion viral protein consisting of or comprising SEQ ID NO:22 or at least 90%, 95%, 96%, 97%, 98% or 99% sequence identity thereto, and the cell receptor protein consisting of or comprising SEQ ID NO:23 or at least 90%, 95%, 96%, 97%, 98% or 99% sequence identity thereto,
k) the prefusion viral protein consisting of or comprising SEQ ID NO:24 or at least 90%, 95%, 96%, 97%, 98% or 99% sequence identity thereto, and the cell receptor protein consisting of or comprising SEQ ID NO:23 or at least 90%, 95%, 96%, 97%, 98% or 99% sequence identity thereto,
l) the prefusion viral protein consisting of or comprising SEQ ID NO:26 or at least 90%, 95%, 96%, 97%, 98% or 99% sequence identity thereto, and the cell receptor protein consisting of or comprising SEQ ID NO:27 or at least 90%, 95%, 96%, 97%, 98% or 99% sequence identity thereto,
m) the prefusion viral protein consisting of or comprising SEQ ID NO:28 or at least 90%, 95%, 96%, 97%, 98% or 99% sequence identity thereto, and the cell receptor protein consisting of or comprising SEQ ID NO:29 or at least 90%, 95%, 96%, 97%, 98% or 99% sequence identity thereto.

In the method of the invention, contacting the prefusion viral protein coupled to the solid support with the agent to provide a mixture is carried out under conditions and during sufficient time, for example about 10 minutes to about 60 minutes, about 20 minutes to 40 minutes, about 30 minutes, to allow binding and/or attachment and/or interaction of the agent to/with the prefusion viral protein.

An embodiment of the invention provides a method for measuring the ability of an agent to block entry of SARS-CoV-2 virus into a cell susceptible to SARS-CoV-2 infection and thereby to inhibit infection of the cell with SARS-CoV-2 virus, comprising
a) providing at least one prefusion SARS-CoV-2 protein, preferably one or more prefusion SARS-CoV-2 protein or at least 90%, 95%, 96%, 97%, 98% or 99% sequence identity thereto, selected from the group comprising SEQ ID NO: 1, 30, 31, 32, 33, 34, 35 and 36, that is stabilized in a prefusion conformation and/or that is reactive with the neutralizing antibodies, and coupling said prefusion SARS-CoV-2 protein to at least one a solid support,
b) contacting the at least one prefusion SARS-CoV-2 protein coupled to the solid support with the agent to provide a mixture,
c) contacting (adding to) the mixture with ACE-2-Fc fusion protein, preferably SEQ ID NO: 2 or at least 90%, 95%, 96%, 97%, 98% or 99% sequence identity thereto, that binds to the prefusion SARS-CoV-2 protein,
e) measuring the binding of the ACE-2-Fc fusion protein to the at least one prefusion SARS-CoV-2 protein to provide an indirect measure of the ability of the agent to inhibit entry of the SARS-CoV-2 virus into the cell.

In a further embodiment of the method of the invention, the at least one prefusion SARS-CoV-2 protein consists of, or comprises, a multiplex of same or different S proteins in prefusion configuration. In a specific embodiment, the multiplex of S protein is a trimer (S³) of the ectodomain in a stabilized prefusion configuration.

The binding of the cell receptor to the prefusion viral protein may be measured by any convenient means known in the art. For example, either the cell receptor or the prefusion viral protein may be labelled with a detectable marker, such as a radioactive label, a fluorescent label, or a marker detectable by way of an enzyme reaction. Many suitable detection systems are known in the art. Thus assay systems which are suitable for use in the method the invention include, but are not limited to, immunoassays such as enzyme linked immunosorbent assay (ELISA); affinity-type assays such as those using coated microtitre plates, beads or slides, or affinity chromatography; fluorescence-activated cell sorting; biosensor assays; and bioassays.

Those skilled in the art will appreciate that the invention described herein is susceptible to variations and modifications other than those specifically described. It is to be understood that the invention includes all such variations and modifications without departing from the spirit or essential characteristics thereof. The invention also includes all of the steps, features, compositions and compounds referred to or indicated in this specification, individually or collectively, and any and all combinations or any two or more of said steps or features. The present disclosure is therefore to be considered as in all aspects illustrated and not restrictive, the scope of the invention being indicated by the appended claims, and all changes which come within the meaning and range of equivalency are intended to be embraced therein.

The foregoing description will be more fully understood with reference to the following Examples. Such Examples, are, however, exemplary of methods of practising the present invention and are not intended to limit the application and the scope of the invention.

### Examples

Study Populations

Seropositive Donors

Cross-validation studies were performed on serum samples identified from the seroprevalence study of the Vaud Canton in Switzerland (SerocoViD) performed by the Centre for Primary Care and Public Health, University of Lausanne (Unisanté), from the Swiss Population based seroprevalence study performed by Coronas Immunitas and from the with hospitalized donor ImmunoCov study performed by the Immunology and Allergy Service, Lausanne University Hospital. The panel of 206 SARS-CoV-2 seropositive samples consists of: 31 hospitalized COVID-19 patients and 64 RT-PCR positive non-hospitalized donors for a total of 95 RT-PCR donors and 111 seropositive donors identified through contact with RT-PCR positive donors, volunteers and asymptomatic or pausisymptomatic donors selected at random from the general population. Serum samples from 59 COVID-19 patients hospitalized with (n=31) or without (n=28) need for stay in the intensive care unit were selected from donors participating in the ImmunoCov study and Unisanté studies.

Pre-COVID-19 Pandemic Donors

Negative control serum samples from 90 adult healthy donors with ages ranging for 18 to 81 years of age were collected prior to November 2019 as part of the Swiss Immune Setpoint study sponsored by Swiss Vaccine Research Institute. Study design and use of subject sera samples were approved by the Institutional Review Board of the Lausanne University Hospital and the ‘Commission d'éthique du Canton de Vaud’ (CER-VD) stated that authorization was not required.

Production of SARS-CoV-2 S Proteins

The S protein trimer was designed to mimic the native trimeric conformation of the protein in vivo and the expression vector was acquired. It encoded the prefusion ectodomain of the original 2019-Coy S protein with a C-terminal T4 foldon fusion domain to stabilize the trimer complex along with C-terminal 8×His and 2×Strep tags for affinity purification. The trimeric Spike protein was transiently expressed in suspension-adapted ExpiCHO cells (Thermo Fisher) in ProCHO5 medium (Lonza) at 5×106 cells/mL using PEI MAX (Polysciences) for DNA delivery. At 1 h post-transfection, dimethyl sulfoxide (DMSO; AppliChem) was added to 2% (v/v). Following a 7-day incubation with agitation at 31° C. and 4.5% CO2, the cell culture medium was harvested and clarified using a 0.22 μm filter. The conditioned medium was loaded onto Streptactin (IBA) and StrepTrap HP (Cytiva) columns in tandem, washed with PBS, and eluted with 10 mM desthiobiotin in PBS. The purity of S protein trimers was determined to be >99% pure by SDS-PAGE analysis. Generation of S protein expression vectors encoding the mutations D614G, D614G plus M153T, N439K, S477N, S477R, E484K, S459Y, N501T, N501Y, K417N, Δ60-70, P681H, Y453F or combinations thereof were generated by InFusion-mediated site directed mutagenesis using primers (not provided herein). The B1.1.7 variant clone was generated by gene synthesis (Twist Biosciences). S proteins for all mutants were produced and purified in an identical manner to the original 2019-Coy S protein.

Coupling of Luminex Beads with SARS-CoV-2 S Protein

Luminex beads used for the serological binding assays were prepared by covalent coupling of SARS-CoV-2 proteins with MagPlex beads using the manufacture's protocol with a Bio-Plex Amine Coupling Kit (Bio-Rad, France). Briefly, 1 ml of MagPlex-C Microspheres (Luminex) were washed with wash buffer and then resuspended in activation buffer containing a freshly prepared solution of 1-ethyl-3-(3-dimethylaminopropyl) carbodiimide (EDC) and N-hydroxysulfosuccinimide (S-NHS), (ThermoFischer, USA). Activated beads were washed in PBS followed by the addition of 50 μg of protein antigen. The coupling reaction was performed at 4° C. overnight with bead agitation using a Hula-Mixer (ThermoFischer). Beads were then washed with PBS, resuspended in blocking buffer then incubated for 30 minutes with agitation at room temperature. Following a final PBS washing step, beads were resuspended in 1.5 ml of storage buffer and kept protected from light in an opaque tube at 4° C. Each of the SARS-CoV-2 proteins was coupled with different colored MagPlex beads so that tests could be performed with a single protein bead per well or in a multiplexed Luminex serological binding assay.

Cell-Free S Protein/ACE2 Surrogate Neutralization Assay

S protein coupled beads are diluted 1/100 in PBS with 50 μl added to each well of a Bio-Plex Pro 96-well Flat Bottom Plates (Bio-Rad). Following bead washing with PBS on a magnetic plate washer (MAG2×program), 80 μl of individual serum samples at different dilutions (e.g. 1/10, 1/30, 1/90, 1/300, 1/2700 and 1/8100) in PBS were added to the plate wells. Control wells were included on each 96-well plate that included beads alone, matching serum dilutions of a control pool of pre-COVID-19 pandemic healthy human sera (BioWest human serum AB males; VWR) and a positive control commercial anti-Spike blocking antibody (SAD-S35 from ACRO Biosciences) or recombinant produced REGN10933 neutralizing antibody discovered and marketed by Regeneron tested in a concentration response. Plates were agitated on a plate shaker for 60 minutes then the ACE2 mouse Fc fusion protein (Creative Biomart or produced by EPFL Protein Production and Structure Core Facility) was then added to each well at a final concentration of 1 μg/ml and agitated for a further 60 minutes. Beads were then washed on the magnetic plate washer and anti-mouse IgG-PE secondary antibody (OneLambda ThermoFisher) was added at a 1/100 dilution with 50 μl per well. Plates were agitated for 45 minutes, washed, the beads resuspended in 8011.1 of reading buffer then read directly on a Bio-Plex 200 plate reader (Bio-Rad). REGN10933 used in this study was a generous gift from Berend-Jan Bosch, Utrecht University, Netherlands. MFI for each of the beads alone wells were averaged and used as the 100% binding signal for the ACE2 receptor to the bead coupled Spike trimer. MFI from the well containing the high concentration (>1 μg/ml) of commercial anti-Spike blocking antibody was used as the maximum inhibition signal. The percent blocking of the S protein trimer/ACE2 interaction was calculated using the formula: % Inhibition=(1−([MFI Test dilution−MFI Max inhibition]/[MFI Max binding−MFI Max inhibition])×100). Serum dilution response inhibition curves were generated with GraphPad Prism 8.3.0 using NonLinear four parameter curve fitting analysis of the log(agonist) vs. response. Sensitivity, specificity and correlative of the tests were calculated with Excel and GraphPad prism.

SARS-CoV-2 Live Virus Cell Based Cytopathic Effect Neutralization Assay

All the biosafety level 3 procedures were approved by the competent Swiss authorities. The day before infection VeroE6 cells were seeded in 96-well plates at a density of 1.25×10E+4 cells per well. Heat inactivated sera from patients were diluted 1:10 in DMEM 2% FCS in a separate 96-well plate. Four-fold dilutions were then prepared in DMEM 2% FCS in a final volume of 60 μl. SARS-CoV-2 (hCoV-19/Switzerland/GE9586) viral stock (2.4×10E6/ml as titrated on VeroE6 cells) diluted 1:100 in DMEM 2% FCS was added to the diluted sera at a 1:1 volume/volume ratio. The virus-serum mixture was incubated at 37° C. for 1 hour then 100 μl of the mixture was subsequently added to the VeroE6 cells in duplicates. After 48 hours of incubation at 37° C. cells were washed once with PBS and fixed with 4% formaldehyde solution for 30 minutes at room temperature. Cells were washed once with PBS and plates were put at 70° C. for 15 minutes for a second inactivation. Staining was performed outside the BSL3 laboratory with 50 μl of 0.1% crystal violet solution for 20 minutes at RT. Wells were washed 3 times with water and plates were dried, scanned and analyzed for the density of live violet stained cells using ImageJ software. For each 96-well plate, at least 4 wells were treated with a negative pool of sera from pre-pandemic healthy donors and 4 wells with virus only and used as negative and positive controls respectively. The percent inhibition of cytopathic effect of the virus was calculated using the formula: % Inhibition=(1−([cell density Test dilution−cell density Max inhibition]/[cell density Max binding−cell density Max inhibition])×100. Serum dilution response inhibition curves were generated with GraphPad Prism 8.3.0 using Non-linear four parameter curve fitting analysis of the log(agonist) vs. response. Sensitivity, specificity and correlative of the tests were calculated with Excel and GraphPad prism. Neutralization IC₅₀ values were calculated as described above for the cell free neutralization assay using the GraphPad prism non-linear four parameter curve fitting analysis.

Spike-Pseudotyped Lentivectors Production and Neutralization Assays

HDM-IDTSpike-fixK plasmid (a kind gift from J. Bloom) encoding for the Wuhan-Hu-1 SARS-Cov2 Spike was modified using QuickChange mutagenesis to generate the D614G mutant and D614G/S477N or D614G/E484K double mutants (primers used are listed in Table 1). Spike-pseudotyped lentivectors were generated by co-transfecting HDM-IDTSpike-fixK, pHAGE2-CMV-Luc-ZSgreen, Hgpm2, REV1b and Tat1b (a kind gift from J. D. Bloom) plasmids into 293T cells for 24 hours with the following ratio 3/9/2/2/2 (18 μg/56.7 cm2 plate) using Fugene transfection reagent (Promega). The following day, cells were transferred in EpiSerf medium, and cell supernatants were collected after 8 hours and 16 hours. Harvested supernatants were pooled, clarified by low-speed centrifugation, filtered to remove cell debris and aliquoted. Lentivector stocks were titrated and normalized for HIV antigen p24 content by ELISA (Zeptometrix).

In the pseudoviral neutralization assay, 293T cells stably expressing the ACE2 receptor were suspended in DMEM medium with 10% FCS and seeded at 1.0×10E+4 cells per well into 96-well plates. After 5 hours in cell culture at 37° C., three-fold dilutions of serum samples were prepared and pre-incubated with the same amount of each pseudovirus in a final volume of 100 μl in DMEM+10% FCS. Following a further 1-hour incubation at 37° C., the pseudoviruses/serum mixture was added to the 293T ACE2 cells. After 48 hours of incubation at 37° C., a luciferase assay was performed to monitor pseudoviral infection, using the ONE-Step™ Luciferase assay system as recommended by the manufacturer (BPS Bioscience). Viral neutralization resulted in the reduction of the relative light units detected. Neutralization IC₅₀ values were calculated as described above for the cell free neutralization and CPE assays using the GraphPad prism non-linear four parameters curve fitting analysis.

Preparation of ACE-2 Protein

ACE-2 protein (hsACE-2, residues S19 to D615) was codon optimized for H. sapiens and cloned into the pTwist_CMV_BetaGlobin_WPRE_Neo (Twist Biosciences). It is preceded with a Human Pregnancy Specific Glycoprotein 1 signal peptide, and C-terminally tagged with a 3C protease cleavage site, twin-strep tag and 10×Histag. The construct was transiently transfected into HEK293 cells (Thermo Fisher) with PEI MAX (Polysciences) in EX-CELL 293 serum-free medium (Sigma), supplemented with 3.75 mM valproic acid. Incubation with agitation was performed at 37° C. and 4.5% CO2 for 8 days. The clarified supernatant was purified in three steps; via a Fastback Ni²⁺ Advance resin (Protein Ark), a Strep-Tactin XT column (IBA Lifesciences) followed by Superdex 200 16/600 column (GE Healthcare) and finally dialyzed into PBS buffer. Average yield for hsACE-2 was 12 mg/L culture.

Neutralization Assay VeroE6 Cells

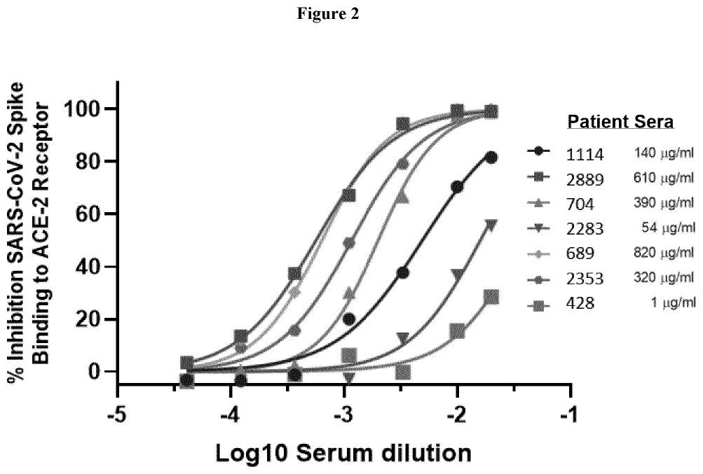

Proof-of-principle was provided by comparing neutralization titers of serum determined in parallel by wild-type SARS-CoV-2 infection of Vero cells and trimeric spike-ACE2 cell-free binding assay (see FIG. 2).

The day before infection VeroE6 cells are seeded in 96-well plates at a density of 1.25×10E+4 cells per well. Heat inactivated sera from patients are diluted 1:10 in DMEM 2% FCS in a separate 96-well plate. Four-fold dilutions are then prepared until 1:40′960 in DMEM 2% FCS in a final volume of 60 μl. SARS-CoV-2 viral stock (2.4×10E6/ml) diluted 1:100 in DMEM 2% FCS was added to the diluted sera at a 1:1 volume/volume ratio. The virus-serum mixture was incubated at 37° C. for 1 h then 100 μl of the mixture was subsequently added to the VeroE6 cells in duplicates. After 48h of incubation at 37° C. cells are washed once with PBS and fixed with 4% formaldehyde solution for 30 min at RT°. Cells are washed twice with PBS and plates are put at 60° C. for 30 min before staining with 50 μl of 0.1% crystal violet solution for 20′ at RT°. Wells are washed twice with water and plates are dried for scanning. A negative pool of sera from pre-pandemic healthy donors was used as negative control. A positive control from a recovered patient with a high titer of neutralizing antibodies is used as a reference. Wells with virus only are used as controls of virus infectivity and cell lysis.

Comparative Test with a Commercially Available In Vitro Cell-Free Assay of Tan, C. W. Et al.

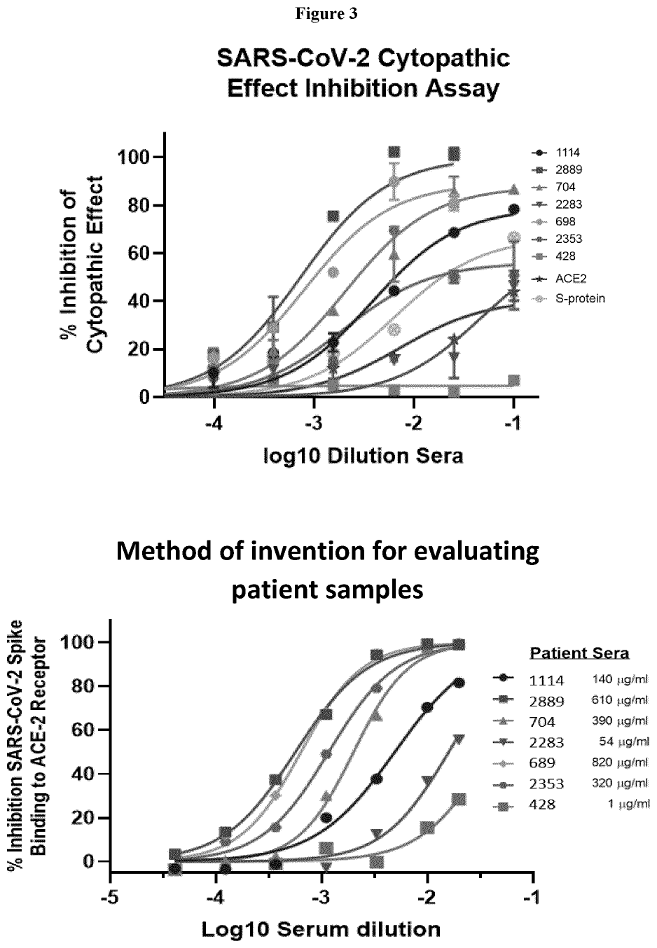

The performance the commercially available in vitro cell-free assay of Tan, C. W. et al. is markedly inferior to that of the invention, notably precluding any quantitative measurement (FIG. 3).

Comparison of Method of the Invention Based on Spike and RBD

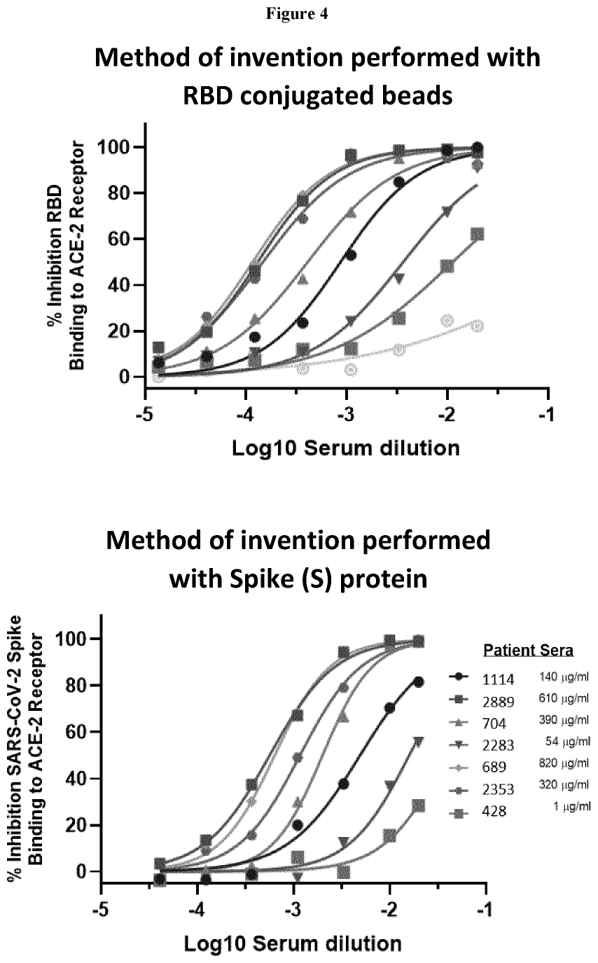

In order to confirm the efficacy of the method of the invention, the method has been carried out with Spike ectodomain (S) protein and with only the receptor binding domain (RBD) peptide. The results (see FIG. 4) show that the method using Spike (S) protein provides much better results and allows quantification of neutralizing antibodies.

Comparison of Method of the Invention (Surrogate Neutralization Assay) with the Commercial Assay from GeneScript

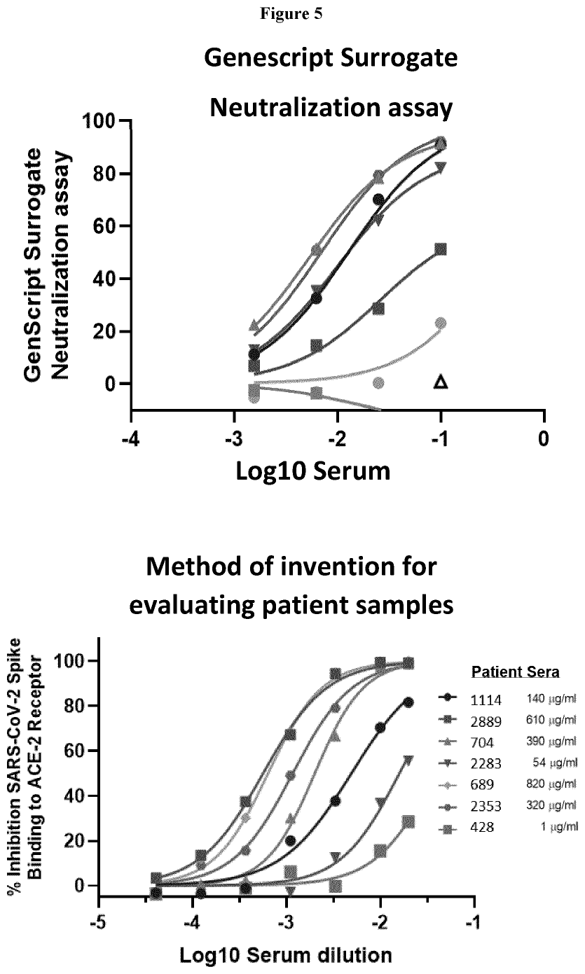

FIG. 5 shows comparison of method of the invention (surrogate neutralization assay) and commercial assay from GeneScript. Commercial assay does not predict the neutralizing potency of patients' sera.

A Cell-Free SARS-CoV-2 S Protein Trimer/ACE2-Based Neutralization Assay

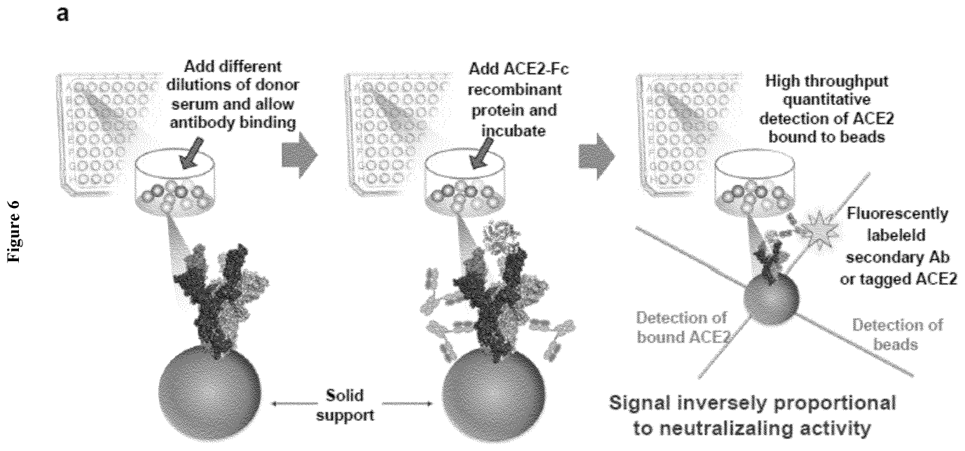

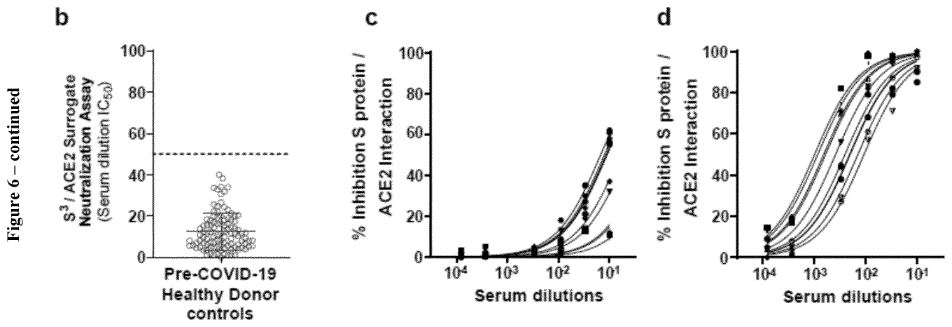

SARS-CoV-2 uses angiotensin-converting enzyme 2 (ACE2) as its primary receptor, which it recognizes via the receptor binding domain (RBD) of its S protein. In return, this 211 amino acid-long region is the main target of neutralizing antibodies. It has been thus hypothesized that an assay (a method) capable of providing a quantitative measurement of the inhibition of SARS-CoV-2 S protein interaction with ACE2 could reveal the neutralizing potential of antibodies found in serum and other biological samples. However, whereas a previously described system based on this assumption relied on the sole use of S protein RBD monomers, according to the present invention it has been reasoned that having the full S protein in trimeric higher order structure, as found on the surface of the virion, was more likely to recapitulate the physiological configuration. Therefore, S protein trimers (S³) has been produced in native prefusion configuration in CHO cells and coupled these proteins to Luminex beads (a solid support). The ability of these S³-bearing beads to recruit a recombinant human ACE2 mouse Fc-tagged fusion protein (ACE2-Fc) has been measured, which was detected with a fluorescently tagged anti-mouse Fc secondary antibody. A standard protocol was then established (FIG. 6a), whereby after S³-carrying beads are first mixed in a 96-well plate with limited dilutions of test serums, before adding ACE2-Fc and pursuing incubation for 60 min in order to reach equilibrium, and finally measuring the amount of captured soluble viral receptor with a fluorescently tagged antibody on a Bio-Plex 200 System. Using this procedure, it was first verified that serum samples from pre-COVID-19 pandemic healthy donors (n=104, FIG. 6b-c) did not significantly cross-react (2) and interfere with ACE2-Fc binding to S³-coupled beads, whereas serum from post-infected donors induced a dilution-dependent signal reduction (FIG. 6d).

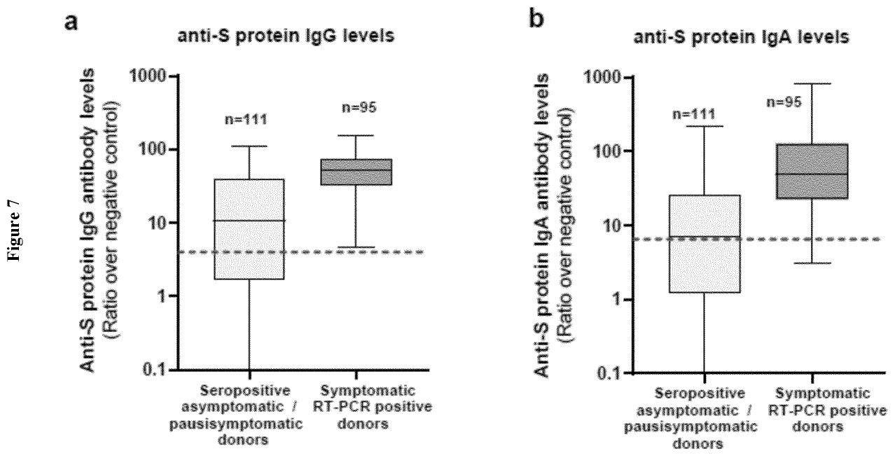

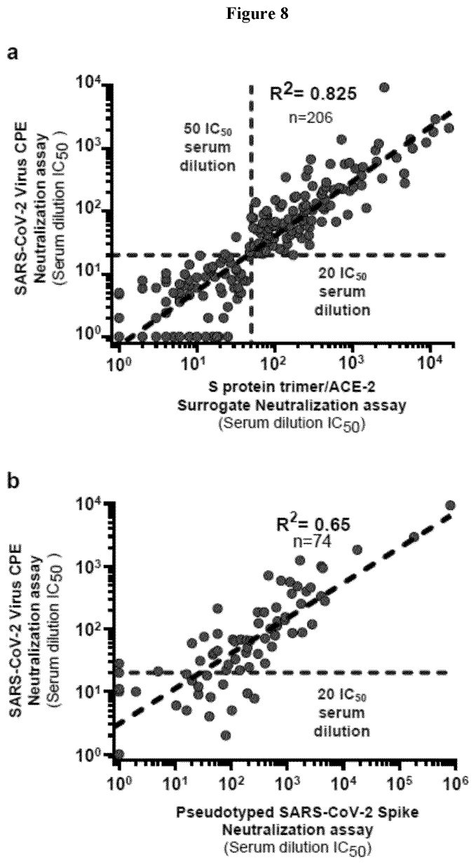

The surrogate neutralization assay was undergone validation with a panel of 206 post-infection serums obtained from individuals, some with a prior history of RT-PCR-documented symptomatic infection requiring or not hospitalization (n=95), and others without known SARS-CoV-2 antecedent but identified as seropositive in a Swiss population serological survey (n=111). As expected, an inverse correlation was noted when comparing these two groups between average serum anti-S protein IgG and/or IgA levels and severity of previous disease (FIG. 7a-b). These 206 serums were tested in parallel with the cell-free S³-ACE2 assay and a conventional live CPE assay in Vero cells. The results revealed a high correlation of the neutralizing titers obtained with the two assays (R2=0.825) over the >3 log range measured amongst the various samples (FIG. 8a). Additionally, a subset of these serums (n=75) was subjected to a LV reporter pseudotype-based neutralization assay, which revealed a weaker correlation with the live virus assay (R2=0.65, n=75; FIG. 8b).

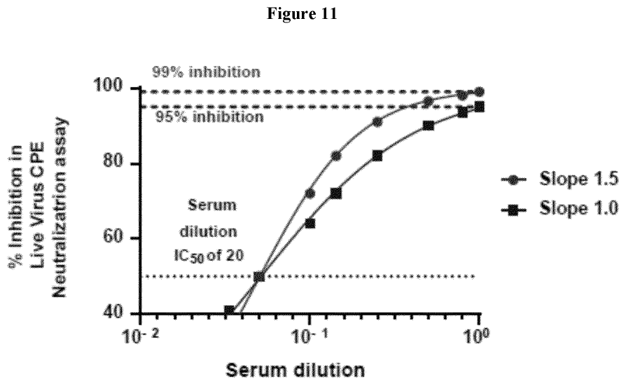

For the S³-ACE2 neutralization assay, a lower limit IC₅₀ serum dilution of 50 was set as the specificity cutoff using IC₅₀ values for the 104 pre-COVID-19 pandemic healthy donor samples (mean IC₅₀ of 12.5+4×9.0 SD; FIG. 6b). A serum dilution IC₅₀ of 20 was selected as the cutoff for positivity in the live SARS-CoV-2 virus CPE assay given that this corresponds to 95-99% viral neutralization (using Nonlinear regression with 1 and 1.5 Hill slopes values to achieve IC95 and IC99, respectively; FIG. 11) for the undiluted serum sample. Using these criteria, the surrogate neutralization assay was determined to achieve 96.7% sensitivity (118 out of 122) and 100% specificity (0 out of 84) relative to the live virus CPE assay (Table 1). Additionally, samples with moderate blocking activity in the S³-ACE2 neutralization assay (serum dilution 1:50 to 1:100) strongly correlated with samples that exhibited weak (1:20 to 1:50) to moderate (1:50 to 1:100) neutralizing activities in the live virus assay.

TABLE 1
Benchmarked performance of the cell-free S³/ACE2
surrogate neutralization assay against the SARS-
CoV-2 live virus CPE neutralization assay

| | | Neutralizing activity in the live virus | |
|---|---|---|---|
| | | CPE assay (IC₅₀ serum dilution) | |
| | | Below lower | |
| | | limit cutoff for | Positive |
| | | neutralizing activity | neutralizing activity |
| | | (<20 IC₅₀; n = 84) | (>20 IC₅₀; n = 122) |
| S³/ACE2 | Below lower | n = 84 S³/ | 3.2% False negative |
| assay (IC₅₀ | limit ACE2 | n = 84 CPE | n = 88 S³/ |
| serum | blocking cutoff | | n = 4 CPE |
| dilution) | (<50 IC₅₀; | | |
| | n = 88) | | |
| | Positive for | 0% False positive | 96.7% Sensitivity |
| | ACE2 blocking | n = 0 S³/ | n = 118 S³/ |
| | activity (>50 | n = 84 CPE | n = 122 CPE |
| | IC₅₀; n = 118) | | |

Multiplexed Measurement of SARS-CoV-2 VOCs Neutralization

A characteristic of Luminex bead-based assays is the use of analytical optics that allow for the simultaneous evaluation of analytes captured by multiple baits, each coupled to beads of different colors. The advantage of this feature has been taken to test in parallel the neutralization potential of post-infection serums against a large array of SARS-CoV-2 S protein variants. For this, a set of S protein derivatives was produced, containing specific mutations alone or in combination, amongst which amino acid changes suspected to contribute to immune escape of some VOCs, such as substitutions at positions K417, E484 and N501 found in the B.1.1.7 (501Y.V1 or UK variant), B.1.351 (501Y.V2 or South African variant) and P.1 strains (501Y.V3 or Brazilian variant) (Table 2)(15, 16, 24).

TABLE 2
S protein mutations in different circulating
SARS-CoV-2 variants of concern

| SARS-CoV-2 | |
|---|---|
| Variant | S protein mutations |
| B.1.1.7/ | Δ69-70, Δ144, N501Y, A570D, D615G, P681H, |
| 501Y.V1 | T716I, S982A, D1118H |
| B.1.351/ | L18F, D80A, D215G, Δ242-244, R246I, K417N, |
| 501Y.V1 | E484K, N501Y, D614G, A701V |
| P.1/501Y.V3 | L18F, T20N, P26S, D138Y, R190S, K417T, E484K, |
| | N501Y, D614G, H655Y, T1027I, V1176F |

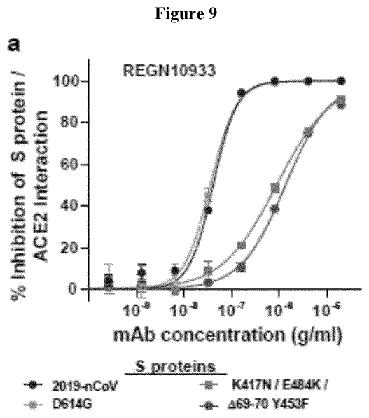

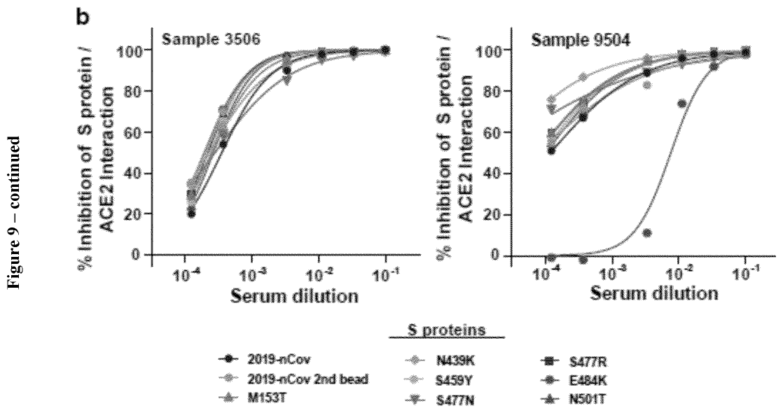

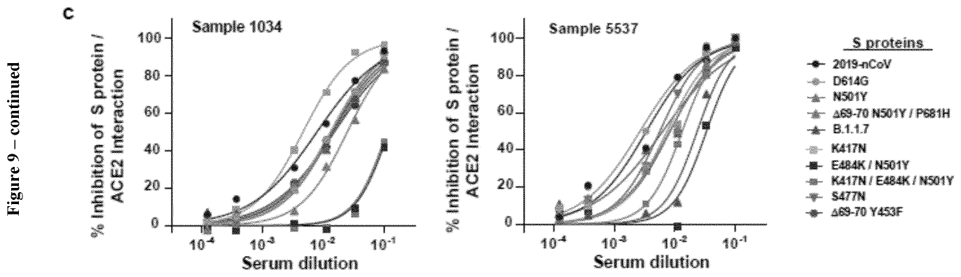

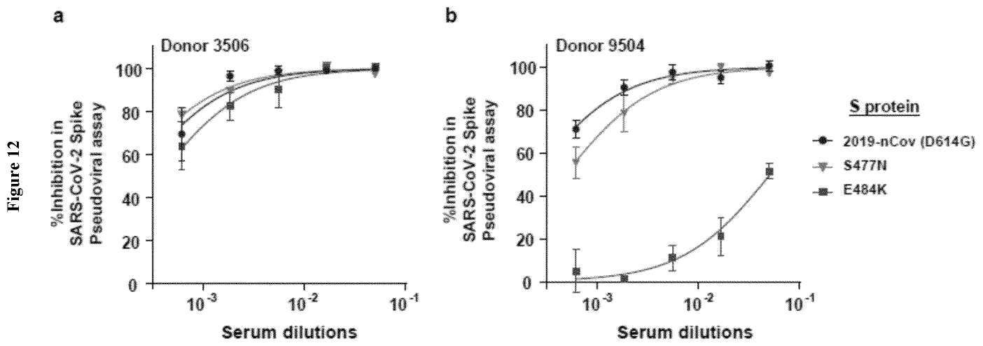

Each of the corresponding S protein trimers were coupled to beads of a given color, and placed equal amounts of each of these beads in the wells of a 96-sell plate. To validate the utility of the method for quantifying virus neutralizing antibodies (S³-ACE2 assay) in identifying S protein mutations conferring reduced sensitivity to neutralization, concentration response curves were generated with the REGN10933 therapeutic antibody profiled against the K417N/E484K/N501Y and Δ69-70 Y453F S protein mutations present in the B.1.351 and mink SARS-CoV-2 variants, respectively (FIG. 9a). Consistent with published results, REGN10933 had a significant loss in potency against S proteins harboring mutations from either the B.1.351 (61-fold loss in IC80 activity) or mink variants (67-fold loss in IC80 activity) (16, 25). The method for quantifying virus neutralizing antibodies (neutralization assay) was performed on a large series of post-infection serums, selected examples of which are illustrated in FIG. 9b-c. In an S protein panel consisting of single amino acid mutations, two donors both exhibited high neutralizing antibody titers (>1,000 IC50) against the original 2019-CoV S protein, as measured in duplicates with beads of two different colors. However, while one (sample 3506) retained full potency against a series of seven S protein point mutants, the other (sample 9504) displayed a >2 log reduced ability to block the interaction of ACE2 with the E484K S protein mutant, a result corroborated in the LV pseudotype reporter assay (FIG. 12).

An additional panel of S³-coupled beads was manufactured to evaluate mutations present in the 501Y lineage circulating SARS-CoV-2 variants, including the so-called B.1.1.7 and B.1.351 or P.1 VOCs (FIG. 7b-c). A representative donor (sample 1034) exhibited neutralizing antibody levels in the higher range of activity (>100 IC₅₀ serum dilution) against the 2019-nCov S protein but only retained moderate blocking activity (50 to 100 IC₅₀ yellow range in FIG. 7c) against most of the singly or multiply mutated S protein derivatives. Importantly, this serum was almost completely inefficient against S proteins containing the E484K mutation found in the B.1.351 and P.1 VOCs (<50 IC₅₀ light red range). Another serum (sample 5537) had a stronger overall neutralization activity but still displayed lower efficacy against S protein derivatives harboring the E484K mutation or against the B.1.1.7 variant (IC₅₀ serum dilutions between 30 and 76).

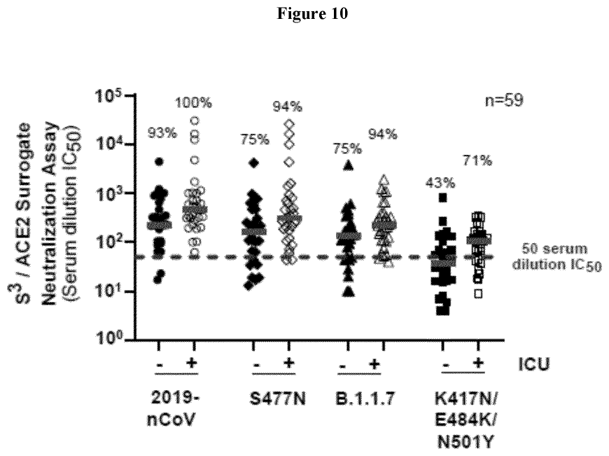

See also FIG. 10, multiplexed analysis of COVID-19 patient serum samples for neutralizing activity against variant of concern S protein mutations

To illustrate the high-throughput screening capability of the method for quantifying virus neutralizing antibodies (S³-ACE2 assay), neutralizing antibody titers against the 2019-CoV and three S protein variants were evaluated in serum from 59 COVID-19 patients hospitalized with (n=31) or without (n=28) need for intensive care unit (ICU) stay (FIG. 8). The majority of patients from both subgroups, all of whom had been infected prior to November 2020, exhibited strong neutralizing activity when profiled against the 2019-nCoV S protein, with 93% of non-ICU and 100% of ICU patients having greater than 50 serum dilution IC₅₀ cut-off. Based on our cross-validation studies, this cut-off value corresponds to antibody levels in undiluted serum samples that would be sufficient to achieve near complete neutralization in the live virus CPE assay (FIG. 11). However, they were less efficient at blocking ACE2 interaction with the S protein found in the B.1.1.7 variant or derivatives containing the S477N substitution or the K417N/E484K/N501Y triple RBD mutation encountered in the B.1.351 variant. Both groups notably exhibited a marked reduction in median IC₅₀ against this latter allele (P<0.0001), with only 58% (43% of non-ICU and 71% of ICU patients) displaying antibody levels above the 50 serum IC₅₀ cut-off. ICU patients additionally presented a weak but significant median increase in neutralizing antibody levels for each of the S proteins tested compared to non-ICU patients (P=0.0127 to 0.0425), consistent with a model whereby more severe and prolonged infections lead to stronger humoral immune responses.

1. A method for quantifying virus neutralizing antibodies in a biological sample of a subject, wherein the virus is an enveloped virus and wherein the method comprises:
a) providing at least one prefusion viral protein(s), that is/are stabilized in a prefusion conformation and that is/are reactive with the virus neutralizing antibodies, and coupling said prefusion viral protein(s) to at least one solid support,
b) contacting the at least one prefusion viral protein(s) coupled to the at least one solid support with the biological sample to provide a mixture,
c) contacting the mixture with one or more cell receptor proteins that bind to the at least one prefusion viral protein(s) or one or more competing antibodies that bind to the at least one prefusion viral protein(s), and
d) detecting and quantifying the virus neutralizing antibodies in the biological sample.

2. The method of claim 1, wherein the method is a multiplex quantifying method of virus neutralizing antibodies, comprising quantifying two or more different virus neutralizing antibodies.

3. The method of claim 1, wherein the virus is a Flavivirus, an Alphavirus, a Coronavirus, a Hepatitis D, an Orthomyxovirus, a Paramyxovirus, a Rhabdovirus, a Bunyavirus, a Filovirus, a Retrovirus, a Herpesvirus, a Poxvirus, a Hepadnavirus, or a Asfarviridae.

4. The method of claim 1, wherein the at least one prefusion viral protein(s) comprises a multiplex of the same prefusion viral proteins or of different prefusion viral proteins.

5. The method of claim 1, wherein the at least one prefusion viral protein(s) comprises a multiplex of S proteins in prefusion configuration.

6. The method of claim 5, wherein the multiplex of S protein is a trimer (S³) in prefusion configuration.

7. The method of claim 1, wherein the at least one prefusion viral protein and the at least one cell receptor protein that binds to the at least one prefusion viral protein are selected from the group consisting of:
a) the prefusion viral protein consisting of or having at least 90% sequence identity to SEQ ID NO:1, 30, 31, 32, 33, 34, 35, or 36, and the cell receptor protein consists of SEQ ID NO:2 or has at least 90% sequence identity thereto,
b) the prefusion viral protein consisting of SEQ ID NO:3 or at least 90% sequence identity thereto, and the cell receptor protein consisting of SEQ ID NO:4 or at least 90% sequence identity thereto,
c) the prefusion viral protein consisting of SEQ ID NO:5 or at least 90% sequence identity thereto, and the cell receptor protein consisting of SEQ ID NO:6 or at least 90% sequence identity thereto,
d) the prefusion viral protein consisting of SEQ ID NO:7 or at least 90% sequence_identity thereto, and the cell receptor protein consisting of SEQ ID NO:8 or at least 90% sequence identity thereto,
e) the prefusion viral protein consisting of SEQ ID NO:9 or at least 90% sequence identity thereto, and the cell receptor protein consisting of SEQ ID NO:10 or at least 90% sequence identity thereto,
f) the prefusion viral protein consisting of SEQ ID NO:12 or at least 90% sequence identity thereto, and the cell receptor protein consisting of SEQ ID NO:13 or at least 90% sequence identity thereto,
g) the prefusion viral protein consisting of SEQ ID NO:14 or SEQ ID NO:15 or at least 90% sequence identity thereto, and the cell receptor protein consisting of SEQ ID NO:16 or at least 90% sequence identity thereto,
h) the prefusion viral protein consisting of SEQ ID NO:17 or at least 90% sequence identity thereto, and the cell receptor protein consisting of SEQ ID NO:18 or at least 90% sequence identity thereto,
i) the prefusion viral protein consisting of SEQ ID NO:19 or at least 90% sequence identity thereto, and the cell receptor protein consisting of SEQ ID NO:20 or at least 90% sequence identity thereto,
j) the prefusion viral protein consisting of SEQ ID NO:22 or at least 90% sequence identity thereto, and the cell receptor protein consisting of SEQ ID NO:23 or at least 90% sequence identity thereto,
k) the prefusion viral protein consisting of SEQ ID NO:24 or at least 90% sequence identity thereto, and the cell receptor protein consisting of SEQ ID NO:23 or at least 90% sequence identity thereto,
l) the prefusion viral protein consisting of SEQ ID NO:26 or at least 90% sequence_identity thereto, and the cell receptor protein consisting of SEQ ID NO:27 or at least 90% sequence identity thereto, and
m) the prefusion viral protein consisting of SEQ ID NO:28 or at least 90% sequence identity thereto, and the cell receptor protein consisting of SEQ ID NO:29 or at least 90% sequence identity thereto.

8. The method of claim 1, wherein the prefusion viral protein consists of or has at least 90% sequence identity to SEQ ID NO: 1, 30, 31, 32, 33, 34, 35, or 36; and the cell receptor protein consists of SEQ ID NO: 2 or has at least 90% sequence identity thereto.

9. The method of claim 1, wherein the virus is a SARS-CoV-2 virus and wherein the at least one prefusion viral protein(s) is/are the at least one prefusion SARS-CoV-2 protein(s), the cell receptor protein is ACE-2-Fc fusion protein, and the one or more competing antibodies bind to the at least one prefusion SARS-CoV-2 protein(s).

10. The method of claim 9, wherein the at least one prefusion SARS-CoV-2 protein(s) is/are two or more prefusion SARS-CoV-2 proteins consisting of or having at least 90% sequence identity to SEQ ID NO: 1, 30, 31, 32, 33, 34, 35 or 36, and the ACE-2-Fc fusion protein consists of SEQ ID NO: 2 or has at least 90% sequence identity thereto.

11. The method of claim 1, wherein the biological sample is a serum sample or a respiratory secretion sample.

12. A kit comprising
at least one prefusion viral protein(s), that is stabilized in a prefusion conformation and that is reactive with neutralizing antibodies,
one or more cell receptor proteins that bind to the at least one prefusion viral protein or one or more competing antibody that binds to the at least one prefusion viral protein(s), and
instructions for use.

13. The kit of claim 12, wherein the prefusion viral protein consists of or has at least 90% sequence identity to SEQ ID NO: 1, SEQ ID NO: 3, SEQ ID NO: 5, SEQ ID NO: 7, SEQ ID NO: 9, SEQ ID NO: 11, SEQ ID NO: 12, SEQ ID NO: 14, SEQ ID NO: 15, SEQ ID NO: 17, SEQ ID NO: 19, SEQ ID NO: 21, SEQ ID NO: 22, SEQ ID NO: 24, SEQ ID NO: 25, SEQ ID NO: 26, SEQ ID NO: 28, SEQ ID NO: 30, SEQ ID NO: 31, SEQ ID NO: 32, SEQ ID NO: 33, SEQ ID NO: 34, SEQ ID NO: 35, or SEQ ID NO: 36 and
the cell receptor protein consists of or has at least 90% sequence identity to SEQ ID NO: 2, SEQ ID NO: 4, SEQ ID NO: 6, SEQ ID NO: 8, SEQ ID NO: 10, SEQ ID NO: 13, SEQ ID NO: 16, SEQ ID NO: 18, SEQ ID NO: 20, SEQ ID NO: 23, SEQ ID NO: 27, or SEQ ID NO: 29.

14. The kit of claim 12, wherein the at least one prefusion viral protein consists of or has at least 90% sequence identity to SEQ ID NO: 1, 30, 31, 32, 33, 34, 35, or 36; and the cell receptor protein consists of SEQ ID NO: 2 or has at least 90% sequence identity thereto.

15. A method for measuring the ability of an agent to block entry of a virus into a cell susceptible to a virus infection and thereby to inhibit infection of the cell with the virus, wherein the virus is an enveloped virus and wherein the method comprises:
a) coupling at least one prefusion viral protein, that is stabilized in a prefusion conformation and/or that is reactive with virus neutralizing antibodies, to at least one solid support,
b) contacting the at least one prefusion viral protein coupled to the at least one solid support with the agent to provide a mixture,
c) contacting the mixture with at least one cell receptor protein that binds to the at least one prefusion viral protein,
d) measuring the binding of the cell receptor to the prefusion viral protein to provide an indirect measure of the ability of the agent to inhibit entry of the virus into the cell.

16. The method of claim 14, wherein the virus is a Flavivirus, a n Alphavirus, a Coronavirus, a Hepatitis D, a Orthomyxovirus, a Paramyxovirus, a Rhabdovirus, a Bunyavirus, a Filovirus, a Retrovirus, a Herpesvirus, a Poxvirus, a Hepadnavirus, or Asfarviridae;

17. The method of claim 14, wherein the virus is a SARS-CoV-2 virus.

18. The method of claim 15, wherein the at least one prefusion viral protein and the at least one cell receptor protein that binds to the at least one prefusion viral protein are selected from the group consisting of:
a) the prefusion viral protein consisting of or having at least 90% sequence identity to SEQ ID NO:1, 30, 31, 32, 33, 34, 35, or 36, and the cell receptor protein consists of SEQ ID NO:2 or at least 90% sequence identity thereto,
b) the prefusion viral protein consisting of SEQ ID NO:3 or at least 90% sequence identity thereto, and the cell receptor protein consisting of SEQ ID NO:4 or at least 90% sequence identity thereto,
c) the prefusion viral protein consisting of SEQ ID NO:5 or at least 90% sequence identity thereto, and the cell receptor protein consisting of SEQ ID NO:6 or at least 90% sequence identity thereto,
d) the prefusion viral protein consisting of SEQ ID NO:7 or at least 90% sequence_identity thereto, and the cell receptor protein consisting of SEQ ID NO:8 or at least 90% sequence identity thereto,
e) the prefusion viral protein consisting of SEQ ID NO:9 or at least 90% sequence identity thereto, and the cell receptor protein consisting of SEQ ID NO:10 or at least 90% sequence identity thereto,
f) the prefusion viral protein consisting of SEQ ID NO:12 or at least 90% sequence identity thereto, and the cell receptor protein consisting of SEQ ID NO:13 or at least 90% sequence identity thereto,
g) the prefusion viral protein consisting of SEQ ID NO:14 or SEQ ID NO:15 or at least 90% sequence identity thereto, and the cell receptor protein consisting of SEQ ID NO:16 or at least 90% sequence identity thereto,
h) the prefusion viral protein consisting of SEQ ID NO:17 or at least 90% sequence identity thereto, and the cell receptor protein consisting of SEQ ID NO:18 or at least 90% sequence identity thereto,
i) the prefusion viral protein consisting of SEQ ID NO:19 or at least 90% sequence identity thereto, and the cell receptor protein consisting of SEQ ID NO:20 or at least 90% sequence identity thereto,
j) the prefusion viral protein consisting of SEQ ID NO:22 or at least 90% sequence_identity thereto, and the cell receptor protein consisting of SEQ ID NO:23 or at least 90%_sequence identity thereto,
k) the prefusion viral protein consisting of SEQ ID NO:24 or at least 90% sequence identity thereto, and the cell receptor protein consisting of SEQ ID NO:23 or at least 90% sequence identity thereto,
l) the prefusion viral protein consisting of SEQ ID NO:26 or at least 90% sequence identity thereto, and the cell receptor protein consisting of SEQ ID NO:27 or at least 90% sequence identity thereto, and
m) the prefusion viral protein consisting of SEQ ID NO:28 or at least 90% sequence identity thereto, and the cell receptor protein consisting of SEQ ID NO:29 or at least 90% sequence identity thereto.

19. The method of claim 15, wherein the prefusion viral protein consists of or has at least 90% sequence identity to SEQ ID NO: 1, 30, 31, 32, 33, 34, 35, or 36; and the cell receptor protein consists of SEQ ID NO: 2 or has at least 90% sequence identity thereto.
